(19) United States
(12) Patent Application Publication
(10) Pub. No.: US 2009/0297550 A1
(43) Pub. Date: Dec. 3, 2009

(54) Chloroplast-derived human vaccine antigens against malaria

(76) Inventors: Henry Daniell, Winter Park, FL (US); Debopam Chakrabarti, Winter Springs, FL (US)

Correspondence Address: ALLEN, DYER, DOPPELT, MILBRATH & GILCHRIST, P.A., 255 SOUTH ORANGE AVENUE, STE. 1401, ORLANDO, FL, 32801, US

(21) Appl. No.: 12/290,509

(22) Filed: Oct. 31, 2008

Related U.S. Application Data
(60) Provisional application No. 60/984,111, filed on Oct. 31, 2007.
(60) Provisional application No. 61/057,442, filed on May 30, 2008.
(60) Provisional application No. 61/091,458, filed on Aug. 25, 2008.

(51) Int. Cl. A61K 39/002; C12N 15/82; A01H 5/00; C07K 14/00; C12P 21/02; A61P 33/06

(52) U.S. Cl. 424/191.1; 800/288; 800/317.3; 435/320.1; 530/403; 435/69.3

(57) ABSTRACT

Disclosed is a method of making a malaria vaccine, the method comprising stably transforming a plant by inserting into its plastid genome a nucleic acid sequence encoding and operable to constitutively express a malaria antigenic polypeptide selected from AMA-1, MSP-1 or both; harvesting the stably transformed plant in whole or in part; purifying the expressed malaria antigenic polypeptide from the harvested plant; and packaging the purified antigenic polypeptide under sterile conditions in an amount for a predetermined dosage. Also disclosed is an oral vaccine effective in raising malaria antibodies in a susceptible host, the vaccine comprising leaf material from an edible plant containing plastids stably transformed to constitutively express a fusion polypeptide consisting essentially of cholera toxin B subunit and a malaria antigenic polypeptide selected from AMA-1, MSP-1 or both.

### RELATED APPLICATIONS

This application claims priority from co-pending provisional application Ser. No. 60/984,111, filed on 31 Oct. 2007, Ser. No. 61/057,442, filed on 30 May 2008, and Ser. No. 61/091,458, which was filed on 25 Aug. 2008, all of which are incorporated herein by reference in their entirety.

### STATEMENT OF GOVERNMENT RIGHTS

The invention claimed herein was made with at least partial support from the U.S. Government. Accordingly, the government may have certain rights in the invention, as specified by law.

### FIELD OF THE INVENTION

The present invention relates to the field of infectious diseases and, more particularly, to the vector-borne disease malaria and to immunogenic malarial antigens expressed in plants.

### BACKGROUND OF THE INVENTION

Malaria is a vector-borne protozoan disease. Four different species of the genus Plasmodium affect humans (P. falciparum, P. vivax, P. malariae and P. ovale) with P. falciparum the most virulent species causing the majority of morbidity and mortality across the world. More than 2 billion people are at risk for malaria with approximately 500 million cases and 1 million deaths annually, mainly in children in sub-Saharan Africa (Greenwood, Fidock et al. 2008; Langhorne, Ndungu et al. 2008). For decades, malaria has remained a prominent public health issue for the international health community and establishment of an effective malaria control program is imperative (Greenwood, Bojang et al. 2005).

### Plasmodium Life Cycle

Malaria parasites multiply in female Anopheles mosquitoes and are transmitted to humans when a mosquito takes a blood meal. When a mosquito pierces the dermis to take a blood meal, Plasmodium sporozoites, alongside saliva, enter the bloodstream, migrate to the liver, and there penetrate hepatocytes, where amplification of the parasite lasts for 2 to 9 days, hence the exoerythocytic cycle (Langhorne, Ndungu et al. 2008).

The parasites differentiate into thousands of merozoites following rupture of liver cells, whereupon the merozoites invade red blood cells (RBCs) and initiate the asexual erythrocytic stage of the life cycle. In the RBCs, the developing parasites appear microscopically as small rings inside a cell in a blood film processed with the appropriate stain, for example, Giemsa stain. The ring stage of the parasite develops a trophozoite within the RBC and finally matures into the schizont stage, which ruptures and releases merozoites in waves of approximately every 48 to 72 hours, depending on the species of Plasmodium (Langhorne, Ndungu et al. 2008). The release of these blood stage parasites is primarily responsible for the clinical manifestations of the disease, such as high fever and shaking chills.

Some of the released parasites develop into sexual erythrocytic male (microgametocytes) and female (macrogametocytes) gametocytes, which upon meeting fuse to form an ookinete when a mosquito takes a blood meal and ingests the gametocytes. The sporogonic cycle begins when the parasites multiply in the mosquito gut. Ookinetes develop into oocysts in the midgut wall of the mosquito. These grow, rupture, and release sporozoites which migrate to the mosquito's salivary glands. They are then ready to infect a new human host to continue the malaria life cycle.

### Clinical Manifestations of Malaria

The most common symptoms of malaria include a flu-like illness with fever, shivering, vomiting, nausea, joint pain, muscle aches, and headaches. The classical symptom of malaria is the cycle of sudden chill with shivering followed by fever and then sweating persisting six to ten hours. The cycle repeats periodically due the release of the asexual erythrocytic stage of the Plasmodium spp. Other symptoms experienced by malaria patients include dizziness, malaise, myalgia, abdominal pain, mild diarrhea, and dry cough. The causative organism of severe malaria is, typically, P. falciparum and consequences include coma and death if untreated. Other complications of severe malaria may occur and include splenomegaly, cerebral ischemia, hepatomegaly, hypoglycemia, hemoglobinuria, renal failure, pulmonary edema, and acidosis. Young children and pregnant women are most vulnerable to severe malaria, along with individuals with no or decreased immunity, a typical example being HIV patients. Severe malaria is considered a medical emergency and should be treated urgently because it can rapidly progress to death within hours or days (Trampuz, Jereb et al. 2003).

### Diagnosis and Treatment of Malaria

The number of cases of malaria is increasing and drug resistance is common, so that prompt diagnosis is essential to reduce morbidity and mortality (Yamey 2004). Clinical diagnosis of patients involves examination of the patient for symptoms but the “gold standard” from a laboratory perspective is examining a blood smear stained with Giemsa stain by microscopic examination (Icke, Davis et al. 2005). If a microscope and staining reagents are not available and lack of quality microscopy, modern antigen detection kits such as a “dipstick” and molecular techniques may be used as alternatives in diagnosis (Greenwood, Bojang et al. 2005) (Icke, Davis et al. 2005). Several issues have arrived with using antigen detection kits and molecular practices such as cost-benefit ratio, accuracy of results, and adequate performance in field conditions.

Malaria must be recognized without delay in order to treat the patient and prevent further disease transmission. Treatment of malaria can be conducted without hospitalization but if severe malaria persists, hospitalization should be advised if possible. Several antimalarial drugs are available for treatment such as chloroquine, sulfadoxine-pyrimethamine, mefloquine, quinine, and doxycycline but combination treatment is ideal. The combination of drugs is preferred because different modes of action are combined to aid in inhibiting the emergence of drug resistant parasites (Greenwood, Bojang et al. 2005). Many factors should be considered when treating a patient with malaria such as the species of infecting parasite, demographic region, cost, pregnancy, pre-existing conditions, and drug allergies.

### Need for a Malaria Vaccine

Preventing mosquito bites with mosquito nets or insect repellents, as well as the spraying of insecticides, can reduce malaria transmission or the need for expensive prophylactic drugs, nevertheless, resurgence of the parasite continues. The causes of resurgence include drug resistance to common antimalarials such as chloroquine, antifolates, sulfadoxine, and artemisin; the mosquito's resistance to widely used insecticides; lack of interest by the pharmaceutical industry in developing new drugs; lack of implementation of effective control measures; increase of tourism; and migration of non-immune populations to malaria endemic areas (Aide, Bassat et al. 2007) (Hyde 2007). A traditional public health tool to effectively reduce the tremendous disease burden would be to develop an efficacious antimalarial vaccine (Doolan and Stewart 2007). Vaccination is one of the most effective means of preventing disease transmission, is cost-effective in reducing new infections, and is easily administered. Many concerns arise when developing an effective vaccine. For example, the complexity of antigens Plasmodium presents throughout the different stages of its life cycle, high polymorphism among parasitic proteins, no appropriate animal model to test the efficacy of a vaccine, high cost of designing a vaccine, and length of vaccine development before it can be marketed by pharmaceutical companies (Aide, Bassat et al. 2007). Currently, there is no licensed effective vaccine for the prevention of malaria. It is hypothesized that a desirable vaccine to prevent malaria progression would contain multi-antigens from different phases of the life cycle.

### Malaria Vaccines are Feasible

There are four main arguments supporting the belief that a malaria vaccine is feasible (Aide, Bassat et al. 2007). Individuals living in endemic areas progressively exhibit naturally acquired immunity by developing partial immunity against severe malaria (Gupta, Snow et al. 1999). Individuals may still become infected with malaria but clinical manifestations and symptoms may be nonexistent due to suppression of parasitemia to undetectable levels (Webster and Hill 2003). Passive transfer of antibodies from either immune malaria patients or maternal transmission during pregnancy has protected patients exposed to the parasite (Sabchareon, Burnouf et al. 1991) or newborn infants (Ballou, Arevalo Herrera et al. 2004), respectively. In the 1970s, experiments were carried out on non-immune volunteers that were exposed to UV irradiated-weakened sporozoites and re-challenged by normal sporozoites with 90% of cases exhibiting short-lived immunity (Rieckmann, Beaudoin et al. 1979). Several studies have reported the efficacy of recent development of protective malaria vaccine candidates in humans (Greenwood, Fidock et al. 2008) (Maher 2008).

### Targets for a Malaria Vaccine

Due to the complexity of the malaria life cycle, vaccines can be targeted to the different stages beginning with the initial exoerythrocyctic stage. The ultimate goal in vaccine development and specific targeting of sporozoites and liver stage parasites is to completely prevent infection (Greenwood, Fidock et al. 2008) by protecting against invasion of hepatocytes or inhibiting parasite development in hepatocytes. Antibodies elicited at this stage would either kill the sporozoite or block hepatocyte invasion. Disrupting parasite development in infected hepatocytes would involve cytotoxic T-lymphocyte mediated lysis. The earliest and now the most advanced pre-erythrocytic studied vaccine candidate utilizes circumsporozoite protein (CSP) as a target because it is the most abundant surface antigen at this stage (Greenwood, Bojang et al. 2005). Current vaccine trials utilizing CSP have designed a hybrid with the hepatitis-B surface antigen and a three-component adjuvant, AS02, known as RTS,S/AS02A but has provided only short-term protection (Greenwood, Bojang et al. 2005). Other antigens in clinical trials include TRAP and LSA but with disappointing results (Maher 2008).

Another strategy in vaccine development could target the second phase of the life cycle, the erythrocytic phase, also known as the asexual blood phase. Vaccines targeted at this stage are designed to prevent disease, not the initial infection, by reducing the number of circulating blood stage parasites (Greenwood, Fidock et al. 2008). The vaccine could either prevent merozoite multiplication or invasion of RBCs, with current research mainly focusing on antigens involved in erythrocyte invasion (Greenwood, Bojang et al. 2005). Antibodies can be elicited to agglutinate merozoites before schizont rupture or to block invasion of RBCs. Current clinical trials are under way looking at several blood stage candidates such as AMA-1, MSP-1, and RESA (Greenwood, Bojang et al. 2005) (Maher 2008).

A final approach in vaccine development is to target the last stage in development, referred as the sexual phase. Vaccines, also known as transmission-blocking, targeted at this stage are important in reducing parasite transmission between hosts by preventing feeding mosquitoes from becoming infected or by interfering with the sexual fusion of gametocytes in the midgut of the mosquito (Greenwood, Bojang et al. 2005) (Saxena, Wu et al. 2007). This is an indirect method of providing protection but it helps in reducing disease transmission in the community (Greenwood, Fidock et al. 2008). Antibodies can be induced to kill gametocytes, to interfere with fertilization of gametocytes, to prevent transformation of the zygote into ookinete, or to hamper egress of ookinetes into viable sporozoites. The approach of using a transmission-blocking vaccine is usually combined with other vaccines targeting other stages (Greenwood, Fidock et al. 2008). Current research on transmission-blocking vaccine candidates include Pfs 25/28, Pfs 48/45, and Pfs 230 (Greenwood, Bojang et al. 2005) (Saxena, Wu et al. 2007) and could play a role in reducing transmission in the population.

### Apical Membrane Antigen-1 (AMA-1)

AMA-1 is a leading asexual blood-stage vaccine candidate (Good, Kaslow et al. 1998) because it plays a crucial role in invasion of Plasmodium parasites. AMA-1 is a type I integral membrane protein (Remarque, Faber et al. 2008) and initially trafficked to micronemes as an 83 kDa precursor protein and proteolytically processed to PfAMA-166 before exportation to the merozoite surface. AMA-1 has been implicated as playing a function in reorienting with the merozoite as the apical organelles and RBC membrane align during invasion (Mitchell, Thomas et al. 2004). Animal and in vitro studies support the crucial role for AMA-1 during invasion of RBCs such as anti-AMA-1 antibodies inhibiting invasion via growth inhibition assays (Hodder, Crewther et al. 2001), antibody-mediated inhibition of antigen processing (Dutta, Haynes et al. 2003), anti-AMA-1 antibodies found in exposed individuals via sero-epidemiological surveys (Thomas, Trape et al. 1994), and AMA-1 has conferred protection in immunization studies (Narum, Ogun et al. 2000). An important issue with the using AMA-1 as a vaccine candidate is it is highly polymorphic (Healer, Murphy et al. 2004) and this reduces susceptibility to the action of inhibitory antibodies. Even though AMA-1 exhibits high polymorphism the C-terminal region is highly conserved and can be blocked by inhibitory antibodies. AMA-1 is not only found in asexual blood stage merozoites but also expressed by sporozoites and liver stage merozoites (Remarque, Faber et al. 2008). Targeting AMA-1 as a vaccine candidate not only can reduce the risk of malaria infection causing clinical disease but also the possibilities of cellular immunity may be stimulated and reduction in exoerythrocytic viability. The current literature definitely supports the idea of AMA-1 and its potential as a vaccine component.

### Merozoite Surface Antigen-1 (MSP-1)

MSP-1 is also another leading asexual blood stage vaccine candidate (Siddiqui, Tam et al. 1987) and is proposed to play a role in parasite invasion of RBCs (Blackman, Heidrich et al. 1990). MSP-1 is a 195 kDa glycoprotein (Mehrizi, Zakeri et al. 2008) found on the merozoite surface, which undergoes two proteolytic cleavages for entry into RBCs. The first cleavage occurs when the merozoite is released from an infected RBC resulting in four polypeptide fragments (83, 30, 38, and 42 kDa) and the second cleavage occurs during invasion of a RBC and involves the C-terminal 42 kDa fragment that is cleaved into 33 and 19 kDa polypeptides (Mehrizi, Zakeri et al. 2008). MSP-1₁₉ stays anchored to the merozoite surface via a GPI tail when RBC invasion takes place (Chenet, Branch et al. 2008). The C-terminal portion of MSP-1₁₉ is a target of some mAb because they inhibit the growth of parasites in vitro (Uthaipibull, Aufiero et al. 2001) and has shown to provide protective immunity (O'Donnell, de Koning-Ward et al. 2001). Vaccines based on the C-terminal region of MSP-1 including MSP-142 and MSP-1₁₉ have provided protection after parasite challenge in Aotus monkeys (Chang, Case et al. 1996) (Kumar, Yadava et al. 1995), antibodies have been shown to inhibit RBC invasion and parasite growth (Chang, Case et al. 1996) (Blackman, Heidrich et al. 1990), and anti-MSP-1₁₉ has been correlated to clinical immunity with reduced parasite numbers and febrile illness (Branch, Udhayakumar et al. 1998). A limiting factor in asexual stage vaccine development is that the C-terminal fragments of MSP-1 parasites isolated in different geographical areas have displayed sequence variation (Mehrizi, Zakeri et al. 2008). Research has provided the insight of using MSP-1 as a potential, promising malaria vaccine antigen.

### SUMMARY OF THE INVENTION

With the foregoing in mind, the present invention advantageously provides malarial antigens expressed in plants via plastid transformation. Preferred plants for use in the invention include tobacco and lettuce, as well as other edible plants. The malarial antigens produced according to the invention were delivered to susceptible subjects by subcutaneous injection or orally by ingestion of minimally processed transplastomic tissue to evaluate their efficacy in eliciting an immune response and protect against malarial infection.

### Preliminary Study

The feasibility of expressing an immunogenic malaria antigen in mice was explored. As described in provisional application Ser. No. 60/984,111, incorporated herein by reference in its entirety, the gene for Merozoite Surface Protein-1 (MSP-1) from the mouse strain of malaria, Plasmodium yoelii, was cloned into a vector effective for transforming the plastid genome in Nicotiana tabacum, the tobacco plant. The C-terminal portion of merozoite surface protein 1 (MSP1) is expressed on the surface of the parasite during the erythrocytic stage, which is considered as a potential vaccine candidate for inhibiting the parasite invasion into RBC. Due to various advantages offered by chloroplast genetic engineering such as hyper-expression of transgene, multigene engineering, absence of position effect and gene silencing, maternal inheritance of transgene etc., PyMSP1₁₉ has been expressed in tobacco via the chloroplast transformation. The site-specific integration of PyMSP1₁₉ gene within chloroplast genome was confirmed by PCR using specific primers and the percentage of homoplasmy vs. heteroplasmy was confirmed by Southern blot. The western blot analysis showed a 17 kDa protein under reducing conditions and the expression levels of PyMSP1₁₉ protein in transgenic lines were up to ˜2% of total soluble protein (TSP) within mature leaves. To test the functionality of chloroplast-derived protein, mice were immunized with the enriched chloroplast-derived PyMSP1₁₉ protein with Freund's adjuvant and they showed 1:7000 antibody titers. The immunized mice were challenged with P. yoelii infected red blood cells (3540% parasitemia) and the percentage parasitemia suggested an inverse correlation with the immune titers.

### The Expanded Study With Tobacco Plants

Transplastomic lines of tobacco plants expressing the malarial antigens fused to the transmucosal carrier Cholera toxin B subunit (CTB-AMA-1) and CTB-MSP-1 were generated. CTB-AMA-1 and CTB-MSP-1 accumulated up to 9.5% and 2% of the total soluble protein, respectively. Chloroplast-derived CTB-AMA-1, CTB-MSP-1, or both antigens were administered to BALB/c mice orally or by subcutaneous injections. The immune response in the experimental animals compared to the control animals was found to be significant. Using an immunofluorescence assay (IFA) and immunoblot, anti-AMA-1 and anti-MSP-1 found in sera of immunized mice recognized native parasite and native parasite protein, respectively. Anti-malarial antibodies inhibited parasite invasion into erythrocytes, as demonstrated by an in vitro parasite inhibition assay. Results of these investigations may lead to a cost-effective malarial vaccine, much needed in developing nations.

### Plants for an Anti-Malarial Vaccine

We believe that there needs to be alternative approach in preparing an effective vaccine to enhance expression levels and potentially protection against malaria infection. The use of other expression systems such as yeast, bacteria, and baculovirus has several disadvantages, such as incorrect folding of recombinant proteins, low yields, and expensive production procedures. Plants could be considered an optimal expression system because they can reduce the cost of purification, processing, cold storage, and delivery (Ruhlman, Ahangari et al. 2007).

The genetic manipulation of tobacco yields large biomass and the success could be extended to edible crops such as carrots, tomatoes, or lettuce. Oral delivery of plant-derived vaccines has been shown to induce both mucosal and systemic immunity (Verma and Daniell 2007) (Nochi, Takagi et al. 2007). The genetic engineering of plants could establish a cost-effective approach in vaccine development for poor, developing countries where malaria infection is most severe.

### Advantages of Chloroplast Genetic Engineering

Many crop species have been genetically modified to express human therapeutic proteins via the nuclear genome but expression levels of the foreign protein are generally insufficient for effective purification and for oral delivery. Chloroplast genetic engineering has been a targeted approach to overcome the concerns of using nuclear transformation such as high level expression of foreign proteins due to thousands of genomes per cell (De Cosa, Moar et al. 2001) (Daniell, Khan et al. 2002), gene containment (Daniell 2002) (Daniell and Parkinson 2003), gene silencing and position effect (De Cosa, Moar et al. 2001), pleiotropic effects (Daniell, Lee et al. 2001), and multi-gene expression (Daniell and Dhingra 2002) (De Cosa, Moar et al. 2001) in a single transformation event. Chloroplast transformation technology has emerged in the advancement in medicine such as expressing proteins in disease resistance (DeGray, Rajasekaran et al. 2001), biopharmaceuticals (Staub, Garcia et al. 2000) (Fernandez-San Millan, Mingo-Castel et al. 2003), and vaccines (Daniell, Streatfield et al. 2001); and in agriculture such as herbicide (Daniell, Datta et al. 1998) and insect resistance (Kota, Daniell et al. 1999), and phytoremediation of toxic metals (Ruiz, Hussein et al. 2003) (Hussein, Ruiz et al. 2007) in transgenic plants. Genetic engineering is achieved by stably integrating the flanking sequences of the foreign gene through homologous recombination with the intergenic regions of the chloroplast genome (Kumar and Daniell 2004). The use of plastid transformation and chloroplast genetic engineering has allowed the expression of foreign genes at a level that is optimal for the oral delivery of vaccines.

### Vaccine Antigens Expressed via the Chloroplast Genome

Several vaccine antigens have been expressed using the approach of chloroplast genetic engineering (Ruhiman, Ahangari et al. 2007). The many advantages of expressing antigens in the chloroplast listed above supports the current rationale of producing transgenic lines expressing the vaccine antigen of interest. Numerous vaccine antigens expressed via the chloroplast are targeted against bacterial, viral, and protozoan pathogens such as the plague F1-V fusion antigen (Arlen, Singleton et al. 2008), Entamoeba histolytica (Chebolu and Daniell 2007) anthrax protective antigen of Bacillus anthracis (Watson, Koya et al. 2004) (Koya, Moayeri et al. 2005), VP6 protein of rotavirus (Birch-Machin, Newell et al. 2004), 2L21 peptide from the virulent canine parvovirus (CPV) (Molina, Hervas-Stubbs et al. 2004), and CTB for cholera (Nochi, Takagi et al. 2007) (Daniell, Lee et al. 2001). The expression levels of the F1-V fusion antigen accumulated up to 14.8% of TSP and after challenge with aerosolized Yersinia pestis, all control animals died in three days and 33% of animals receiving boosts of subcutaneous injections of F1-V and 88% of mice receiving oral boosts of F1-V were protected (Arlen, Singleton et al. 2008). This finding brings hope of utilizing the approach of chloroplast technology and oral-deliverable, cost-effective vaccines closer to reality.

### Cholera Toxin B Subunit (CTB)

The Gram-negative bacterium Vibrio cholerae secretes an enterotoxin known as cholera toxin (CT). CT is oligomer made up of six proteins AB5 consisting of one toxic 27 kDa A subunit and five non-toxic B subunits each weighing 11.6 kDa (Daniell, Lee et al. 2001). This hexameric complex facilitates entry into the mucosal epithelium of the intestine via cholera toxin B subunit (CTB) and the GM1 ganglioside receptors (Daniell, Lee et al. 2001). GM1 gangliosides are found on the gut epithelial surface and it is known for CTB to have a high affinity to these glycosphingolipids (Mor, Gomez-Lim et al. 1998). CTB is known, when given orally, to be a safe, potent, mucosal immunogen and adjuvant (Holmgren, Lycke et al. 1993). CTB has the potential to enhance the immune response when coupled to other pathogenic antigens (Daniell, Lee et al. 2001). A previous study used CTB-GFP plants and orally administered the transgenic leaf material to mice and observed CTB in the intestinal wall and GFP fluorescence in mouse intestinal mucosa, liver, and spleen (Limaye, Koya et al. 2006). This opens the possibility of creating orally deliverable human therapeutic proteins effective via receptor-mediated intestinal absorption (Ruhiman, Ahangari et al. 2007).

### Bioencapsulation for Oral Delivery

An issue of concern arises with the oral delivery of vaccine antigens into the body. The antigen delivered needs to be intact and retain its biological activity and withstand the digestive enzymes present in the stomach. Bioencapsulation is the term applied to the ability of plant cells to enclose and thereby protect an orally delivered protein from acid digestion (Walmsley and Amtzen 2000). Orally deliverable vaccine proteins need to cross the mucosal barrier effectively to provide protection in the event of the immune system encountering the pathogen. Previous reports show that GFP was bioencapsulated via receptor-mediated oral delivery and the utilization of the transmucosal carrier CTB (Limaye, Koya et al. 2006). GFP and not CTB was present in the liver of mice after oral delivery indicating it was protected from the digestive enzymes of the stomach. The roles of receptor-mediated oral delivery and bioencapsulation provide insight in producing low-cost vaccines that deliver vaccine antigens effectively.

### The Present Invention

With the foregoing in mind, the present invention discloses a method of producing malaria antigens in a plant, the method comprising stably transforming the plant by inserting into its plastid genome a nucleic acid sequence encoding and operable to express a malaria antigenic polypeptide selected from AMA-1, MSP-1 or both. “Stably transformed” means that the integrated DNA sequences are inherited through plastid genome replication by daughter cells or organisms. This stability is exhibited by the ability to establish permanent cell lines, clones, or transgenic plants comprised of a population containing the exogenous DNA.

The method of the invention also includes treating a host susceptible to malaria by administering to the host the malaria antigenic polypeptides produced in plants by a route effective for eliciting an antibody response. The method of the invention further includes an orally deliverable vaccine effective for raising a malaria antibody response in the vaccinated individual.

Moreover, the invention also includes an expression cassette effective for stably transforming a plant plastid genome to express one or more malaria antigenic polypeptides. The cassette comprises a nucleic acid sequence including two untranslated flanking regions homologous to parts of and effective for integrating into the plastid genome, and between the flanking regions a region encoding a malaria antigenic polypeptide selected from AMA-1, MSP-1 and combinations thereof, a region encoding a marker conferring resistance to a selective agent and a promoter region effective for constitutive expression of at least the malaria antigenic polypeptide and the resistance marker. The expression cassette preferably has between the flanking regions a region encoding cholera toxin B subunit, such that an expressed malaria antigenic polypeptide is a fusion polypeptide therewith. Also included in the invention is the fusion polypeptide expressed by the cassette and in a form purified from the transformed plant and the transformed plant itself containing the plastid genome stably transformed with the cassette, and its cuttings, seeds and progeny.

The invention further includes an oral vaccine effective in raising malaria antibodies in a susceptible host, the vaccine comprising leaf material from an edible plant containing plastids stably transformed to constitutively express a fusion polypeptide consisting essentially of cholera toxin B subunit and a malaria antigenic polypeptide selected from AMA-1, MSP-1 or both. Part of the invention includes a method of treating a host susceptible to malaria, the method comprising orally administering the vaccine of claim 14.

Yet additionally disclosed herein is a method of making a malaria vaccine, the method comprising stably transforming a plant by inserting into its plastid genome a nucleic acid sequence encoding and operable to constitutively express a malaria antigenic polypeptide selected from AMA-1, MSP-1 or both. The method continues by harvesting the stably transformed plant in whole or in part, purifying the expressed malaria antigenic polypeptide from the harvested plant, and packaging the purified antigenic polypeptide under sterile conditions in an amount for a predetermined dosage. A preferred plant for use in the method is a species of the genus Nicotiana, and most preferably is a variety of the species Nicotiana tabacum.

The method of the invention further includes a method of making an oral malaria vaccine by stably transforming an edible plant by inserting into its plastid genome a nucleic acid sequence encoding and operable to constitutively express a malaria antigenic polypeptide selected from AMA-1, MSP-1 or both, harvesting the stably transformed edible plant or parts thereof, and packaging the harvest for oral consumption. The harvest is preferably packaged in dried form.

### BRIEF DESCRIPTION OF THE DRAWINGS

Some of the features, advantages, and benefits of the present invention having been stated, others will become apparent as the description proceeds when taken in conjunction with the accompanying drawings, presented for solely for exemplary purposes and not with intent to limit the invention thereto, and in which:

FIG. 1 is a schematic diagram showing “Chloroplast pLD-UTR CTB-Malarial Antigens”, and demonstrates the proposed orientation of the transgene into the chloroplast vector according to an embodiment of the present invention;

FIG. 2 shows the PCR Analysis of CTB, FC AMA-1, and MSP-1;

FIG. 3 illustrates the Analysis of Cloning CTB FC AMA-1 and CTB MSP-1 Into the pLD-UTR Chloroplast Vector;

FIG. 4 depicts the PCR Analysis of Wild Type and Positive Transformants;

FIG. 5 is the Evaluation of Transgene Integration into the Chloroplast Genome of Homoplasmic Plants by Southern Blot;

FIG. 6 shows various generations of transgenic plants, (A) being after four to five weeks following particle bombardment, (B) shoots appearing within two to three weeks, and (C) homoplasmic plants after being transferred to a greenhouse;

FIG. 7 shows the Immunoblot Analysis to Confirm Expression of CTB-Malaria Antigens in Nicotiana tabacum Crude Extracts;

FIG. 8 shows graphs illustrating the Quantification of Chloroplast-Derived CTB-Malarial Expression;

FIG. 9 is a gel separation showing Increased Resolution of Chloroplast-Derived CTB FC-AMA-1 Protein After Talon Purification;

FIG. 10 depicts an Immunoblot Analysis of Enrichment of Malarial Antigens from Nicotiana tabacum Extracts;

FIG. 11 is an Immunoblot of the Eluted Protein Fractions were Analyzed and Compared to Known Quantities of CTB Protein;

FIG. 12 shows an immunoblot confirming Recognition of Native Parasite Protein by Anti-AMA-1 and Anti-MSP1 Antibodies;

FIG. 13 presents visible and immunofluorescence photomicrographs showing recognition of native parasite by Anti-AMA-1 and Anti-MSP-1 antibodies;

FIG. 14 shows four photomicrographs of blood smears for evaluating level of parasitemia in the several groups of treated mice;

FIG. 15 is a diagram map of the lettuce chloroplast transformation vector and the site of transgene integration into targeting vector; restriction site for Southern blot analysis; (a) map of plasmid pLsDV CTB-AMA1 vector showing flanking sequence, promoter, selectable marker gene cassette and CTB-AMA1 protein expressing cassette with restriction sites used Southern analysis; (b) layout of plasmid pLsDV CTB-MSP1 vector showing, flanking sequence, promoter, selectable marker gene cassette and CTB-MSP1 protein expression cassette;

FIG. 16 analysis showing all six resistant shoots from pLsDV CTB-AMA-1 and five from pLsDV CTB-MSP-1 being PCR positive for chloroplast transgenic lines;

FIG. 17 shows that all pLsDV CTB-AMA-1 and pLsDV CTB-MSP-1 transgenic plants from the third round of selection showed homoplasmy;

FIGS. 18 and 19: immunoblots were performed on transgenic lines containing CTB-AMA-1 and CTB-MSP-1 transgene; immunodetection with CTB polyclonal antibody showed 27.5 kDa of CTB fused polypeptide on CTB AMA-1 blots (FIGS. 18 and 19)); large amount of protein could be detected in pellet in FIG. 19;

FIG. 20 is an immunoblot at in FIGS. 18 and 19, but showing a 23 kDa of CTB fused polypeptide on CTB MSP-1 blots; formation of dimers, trimers, tetramers and pentamers of the CTB-AMA1 and CTB-MSP1 fusion protein was observed;

FIG. 21 is a bar graph showing that CTB-AMA-1 and CTB-MSP-1 protein expression level of tobacco matured leaves reached 12.3% and 8% of the TSP respectively; whereas, in lettuce the CTB-AMA-1 and CTB-MSP-1 protein expression level reached 9.4% and 4.8% of the TSP respectively under the green-house growth conditions;

FIG. 22 shows production of chloroplast transformed lettuce (FIG. 9): spectinomycin resistance shoots obtained from the bombarded leaf after three weeks of selection; leaves from the resistance shoots were excised into 0.5 cm2 and subcultured on LR medium for second round of selection to obtain homoplasmic shoots; derived shoots were then transferred on to LD medium for rooting. Homoplasmic plants confirmed by southern analysis were transferred to jiffy pots for acclimatization; healthy and hardened plants were transferred to the green house; matured plants produced normal inflorescence and seeds; and

FIG. 23 shows germination of wild-type and transformed seeds to show cytoplasmic inheritance of transgene and no Mendelian segregation.

### DETAILED DESCRIPTION OF THE PREFERRED EMBODIMENT

The present invention will now be described more fully hereinafter with reference to the accompanying drawings, in which preferred embodiments of the invention are shown. Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. Any publications, patent applications, patents, and other references mentioned herein are incorporated by reference in their entirety. In case of conflict, the present specification, including any definitions, will control. In addition, the materials, methods and examples given are illustrative in nature only and not intended to be limiting. Accordingly, this invention may be embodied in many different forms and should not be construed as limited to the illustrated embodiments set forth herein. Rather, these illustrated embodiments are provided so that this disclosure will be thorough and complete, and will fully convey the scope of the invention to those skilled in the art. Other features and advantages of the invention will be apparent from the following detailed description, and from the claims.

### Acronyms and Abbreviations Used

aadA—Aminoglycoside 3′ adenosyl transferase

### APS—Ammonium Persulfate

### BAP—Benzylaminopurin

### BME—Beta-mercaptoethanol

### BSA—Bovine Serum Albumin

### CSP—Circumsporozoite Protein

### CT—Cholera Toxin

### CTB—Cholera Toxin B Subunit

### CTAB—Cetyltrimethylammonium Bromide

DABCO—1,4-diazabicyclo[2,2,2]octane

### EDTA—Ethylene Diamine Tetra-Acetic Acid

### ELISA—Enzyme Linked Immunosorbent Assay

### FC—Furin Cleavage Site

### GFP—Green Fluorescent Protein

### GPI—Glycosylphosphatidylinositol

### IFA—Immunofluorescence Assay

### LSA—Liver Stage Antigen

### NaBH4—Sodium Borohydride

### NaCl—Sodium Chloride

### NAA—α-Naphtalene Acetic Acid

### PBS—Phosphate Buffered Saline

pBSK+—pBlueScript SK+

### PBS-T—Phosphate Buffered Saline—Tween

### PCR—Polymerase Chain Reaction

### PEI—Polyethylenimine

Pf—Plasmodium falciparum
PfAMA1—Plasmodium falciparum Apical Membrane Antigen 1
PfMSP1₁₉—Plasmodium falciparum Merozoite Surface Antigen 1₁₉
psbA—Photosystem b/A

### PTM—Phosphate Buffered Saline—Tween—Milk

### RBC—Red Blood Cells

### RESA—Ring-infected Erythrocyte Surface Antigen

### RT-PCR—Reverse Transcriptase—Polymerase Chain Reaction

### SDS—Sodium Dodecyl Sulfate

### SDS-PAGE—Sodium Dodecyl Sulfate—Polyacrylamide Gel Electrophoresis

### TEMED—N,N,N,N′-Tetra-Methyl-Ethylene Diamine

### TRAP—Thrombospondin-Related Anonymous Protein

### TSP—Total Soluble Protein UTR—Untranslated Region UV—Ultraviolet

### Rationale and Approach

A major objective of this project was to express and characterize the malarial antigens CTB-AMA-1 and CTB-MSP-1 via the chloroplast genome and evaluate the immunogenicity of the chloroplast derived antigens. To achieve these objectives, malarial gene products were confirmed and cloned into the plastid vector designated as pLD-UTR (according to U.S. Pat. No. 7,129,391 which is incorporated herein by reference in its entirety). Using particle bombardment, transgenic lines were obtained to express the chloroplast-derived malarial antigens. Confirmation of expression and quantification of the malarial proteins allowed the transgenic plants to be orally delivered to rodents via oral gavage. The malarial antigens were enriched for the comparison of subcutaneous injection versus oral delivery. The immunogenicity of the purified antigens was evaluated by determining mouse antibody titers via enzyme-linked immunosorbent assays (ELISAs). If the antigens were immunogenic, they were then tested to determine their ability inhibit parasite invasion into RBCs.

### Materials and Methods

### Amplification of AMA-1 and MSP-1 in Asexual Stages and Cloning

Based on the C-terminal nucleotide sequences of AMA-1 and MSP-1, forward and reverse primers were designed to amplify the gene. Forward primers (5′-CCGCTCGAGCATATGGCTTTGTCCCATCCCAT-3′; SEQ ID NO:1) with an XhoI site; and (5′-CCGAATTCGGACCAGGACCAATTTCACAACACCAATGA-3′; SEQ ID NO:2) with an EcoRI site for AMA-1 and MSP-1, were made respectively. The reverse primers were (5′-CGGAATTCTTTCATGTTATCATAAGTTG-3′; SEQ ID NO:3) designed with an EcoRI site and (5′-ATAAGAATGCGGCCGCTTAGTTAGAGGAACTGCAGAAAATAC-3′; SEQ ID NO:4) designed with a NotI site for AMA-1 and MSP-1, respectively.

The total RNA used for RT-PCR was isolated prior (stored at −80° C.) by harvesting an asynchronous 3D7 P. falciparum culture with 0.05% saponin lysis and isolating total RNA from the parasite pellet by using the RNAgents Total RNA Isolation System (Promega). The StrataScipt one-tube RT-PCR system (Stratagene) with Easy-ATM High-Fidelity PCR Cloning Enzyme was used to reverse transcribe and amplify the genes using 200 ng of total RNA and the gene-specific primers. RTPCR cycling conditions were as follows: 1 cycle of reverse transcription at 42° C. for 30 minutes followed by transcriptase enzyme inactivation at 95° C. for 30 seconds; 5 cycles of denaturation at 95° C. for 30 seconds, annealing at 50° C. for 30 seconds and extension at 68° C. for 6 minutes; 35 cycles at 95° C. for 30 seconds, 58° C. for 30 seconds and 65° C. for 6 minutes and 1 cycle at 65° C. for 10 minutes. 5 μL of RT-PCR products were analyzed by electrophoresis on a 0.8% agarose gel and visualized by the Gel Doc 2000 (Bio Rad). The remainder of the RT-PCR product was purified with the QIAquick PCR Purification Kit (Qiagen) and confirmed by a 0.8% agarose gel. The genes were cloned into the pGEMT Easy Vector (Promega) and confirmed by digestion with EcoRI and gel electrophoresis. The DNA was sent to the University of Florida: DNA Sequencing (ICBR: The Biotechnology Program) and by using the DNA STAR SeqMan program the sequence was confirmed. By using the template DNA and new primers containing the following restriction sites: 5′ primer with SmaI and 3′ primer with NotI, AMA-1 (also contained a furin cleavage site: Arg-Lys-Lys-Arg at the 5′ end) and MSP-1 genes were amplified by PCR and confirmed by a 0.8% agarose gel. The FC AMA-1 and MSP-1 genes were subcloned into the pBSK+ (Stratagene) vector.

### Amplification and Cloning of CTB

Template DNA containing the CTB gene was provided by Dr. Daniell's lab. The CTB gene was amplified via PCR with a 5′ primer with XhoI and NdeI sites and a 3′ primer with a SmaI restriction site. The CTB gene was cloned into the pGEMT Easy Vector (Promega) and the sequence was confirmed. CTB was subcloned into the pBSK+ vector using the XhoI and SmaI sites. The pBSK+CTB, pBSK+FC AMA-1, and pBSK+MSP-1 were digested with SmaI and NotI restriction enzymes and the FC AMA-1 and MSP-1 genes were ligated into the pBSK+CTB plasmid to complete the fusion genes: CTB FC AMA-1 and CTB MSP-1.

### Chloroplast Plasmid Construction

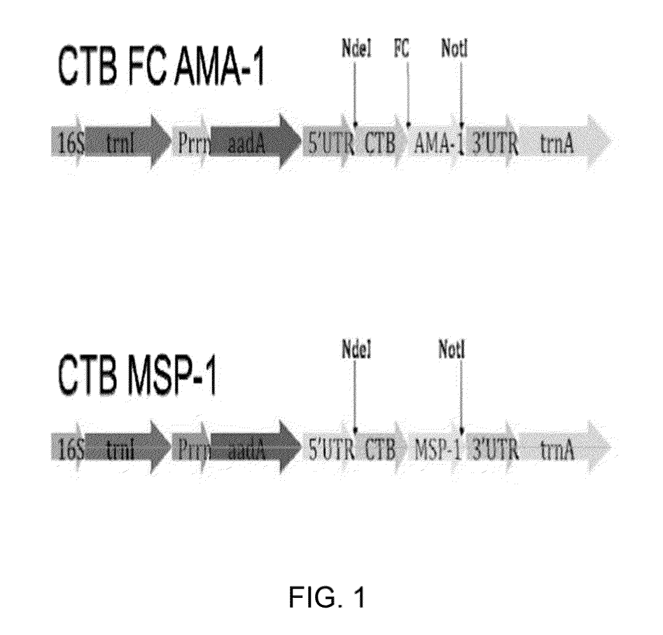

The CTB FC AMA-1 and CTB MSP-1 transgenes in the pBSK+ vector were cut with NdeI and NotI restriction enzymes and ligated into the pLD-UTR vector with T4 ligase (New England BioLabs) and transformed into supercompetent E. coli XL-10 Gold cells. FIG. 1, captioned Chloroplast pLD-UTR CTB-Malarial Antigens, demonstrates the proposed orientation of the transgene into the chloroplast vector.

After transformation, colonies were selected and grown overnight in 5 mL of LB broth and 5 μL of ampillicin (100 mg/mL). The DNA was isolated by using the QIAprep Spin Miniprep Kit (Qiagen), digested with NdeI and NotI restriction enzymes, and analyzed by a 0.8% agarose gel. From the glycerol stock containing a positive clone, LB 100 mg/mL ampillicin plates were streaked and a colony was grown overnight. The chloroplast plasmid DNA was purified with the Qiagen Plasmid Maxi Kit and the DNA was analyzed by gel electrophoresis on a 0.8% agarose gel.

### Transformation and Regeneration of Transgenic Plants

Preparation of Gold Particles and Coating with DNA

A mixture of 50 mg of gold particles and 1 mL of 100% ethanol was vortexed for two minutes and centrifuged at 10,000×g for three minutes in a 1.5 mL Eppendorf tube. The supernatant was discarded and the gold particles were resuspended in 1 mL of 70% ethanol for one minute. The suspension was left at room temperature for fifteen minutes with mixing intermittently. The gold particles were pelleted by centrifuging at 5,000×g for two minutes and the supernatant was discarded. The gold particles were vortexed with 1 mL of sterile distilled water and incubated at room temperature for one minute and centrifuged at 5,000×g for two minutes. The steps to wash the gold particles with sterile water were repeated three times (Kumar and Daniell 2004). The gold particles were resuspended in 1 mL of sterile 50% glycerol and stored on ice until use.

50 μL of gold particles was removed from the stock and transferred into a 1.5 mL microcentrifuge tube along with 10 μg of plasmid DNA. To ensure proper binding of DNA to the gold particles 50 μL of 2.5 M CaCl2 and 20 μL of 0.1 M spermidine-free base was added. The mixture was vortexed for twenty minutes at 4° C. and the DNA-coated gold particles were centrifuged at 10,000×g for one minute. The supernatant was removed and the pellet was washed four times in 200 μL of absolute alcohol. The DNA-coated gold particles were resuspended in 50 μL of 100% ethanol. An aliquot of 10 μL of vortexed DNA-coated gold particles were loaded onto sterile macrocarriers and allowed to air dry in the laminar air flow hood.

### Bombardment of Leaf Tissue

The bombardment was performed under sterile conditions with all equipment, including the gene gun (Bio-Rad PDS-1000/He), sterilized with 95% ethanol. Green healthy leaves from the in vitro tobacco plant, Nicotiana tabacum variety Petit Havana, were cut from young plants and placed with the adaxial side facing up on autoclaved Whatman filter paper on solidified RMOP medium. The gun was loaded with the DNA-gold coated particles and the bombardment was performed at 1,100 psi and 28 Hg. After the bombardment, the potentially transformed leaves were covered with aluminum foil and kept in the dark for 48 hours at room temperature (Kumar and Daniell 2004).

### Regeneration and Selection of Transplastomic Shoots

After 48 hours, the bombarded leaves were cut into approximately 5×5 mm² pieces and transferred to RMOP media (one pack of MS basal salt mixture, 30 g of sucrose, 100 mg myoinositol, 1 mL of 1 mg/mL BAP, 100 μL of 1 mg/mL NAA, 1 mL of thiamine hydrochloride to 1 L of sterile distilled water and adjusted to pH 5.8 using 1N KOH; 6 g of phytagar was added to media for solidification; autoclaved and cooled before pouring into Petri dishes) containing 500 μg/mL of spectinomycin with the bombarded side in contact with the medium. The Petri dish was sealed with parafilm and kept in the culture room until putative transgenic shoots appear. Confirmed positive transgenic lines by PCR analysis were subjected to a second round of selection to achieve homoplasmy. After four weeks of secondary selection, the shoots were transferred to MSO media (one packet of MS basal salt mixture and 30 g of sucrose to 1 L of sterile distilled water; prepared to pH of 5.8 using 1 N KOH; 6 g of phytagar) with 500 μg/mL spectinomycin. This accounts for the third round of selection and rooting.

### Isolation of Plant DNA Confirmation of Transgene Integration

Before proceeding to the next round of selection, 100 mg of leaf material was harvested from putative transplastomic shoots. The Qiagen DNeasy Plant Mini Kit was used to isolate plant genomic DNA, following the manufacturer's protocol. The procedure yields approximately 20-30 μg of DNA and the isolated DNA was used for PCR analysis. PCR was used to confirm transgene cassette integration into the chloroplast genome by the primer pair 3P (5′AAAACCCGTCCTCAGTTCGGATTGC-3′; SEQ ID NO:5) and 3M (5′CCGCGTTGTTTCATCAAGCCTTACG-3′; SEQ ID NO:6) (Daniell, Ruiz et al. 2005). The integration of the gene of interest was confirmed by PCR using the primer pair 5P (5′-CTGTAGAAGTCACCATTGTTGTGC-3′; SEQ ID NO:7) and 2M (5′-TGACTGCCCACCTGAGAGCGGACA-3′; SEQ ID NO:8) (Daniell, Ruiz et al. 2005). DNA isolated from wild type Petit Havana was used as the negative control and DNA isolated from known transgenic plant material was used as the positive control. For PCR analysis, 50 μL of reaction volume was prepared in a 0.2 mL PCR tube: 1 μL of 100 ng/μL genomic DNA, 5 μL of 10×PCR reaction buffer, 4 μL of 2.5 mM dNTP, 1 μL of 3P and 3M primers (or 5P and 2M primers), 1 μL of Taq DNA polymerase, and sterile distilled water to make up the total volume. The initial denaturation was set at 94° for 5 minutes and amplification was carried out for thirty cycles of the following program: 94° C. for 1 minute (denaturation), 60° C. for one minute (annealing), and 72° C. for 2 minutes (extension). Final extension of ten minutes at 72° C. was carried at the end of PCR. To examine the PCR product via agarose gel electrophoresis, 5 μL of it was loaded, along with controls, into a 0.8% agarose gel and visualized by the gel doc.

### Southern Blot Analysis

### Isolation of Plant Genomic DNA

Leaf material from transgenic and non-transgenic plants was removed with aseptic technique from the in vitro greenhouse and ground with liquid nitrogen into 100 mg of a fine powder. Previously made and stored at 65° C., 1 mL of DNA extraction buffer (Tris-HCl pH8, EDTA, NaCl, CTAB, BME up to 10 mL of water) was added to the leaf material and incubated at 65° C. for thirty minutes with gentle mixing every five to eight minutes. An aliquot of 667 μL of (48:2) chloroform:isoamyl alcohol was added to the homogenate with gentle inverting for one minute followed by centrifugation at 10,000 rpm for ten minutes. The supernatant was collected and placed into new tubes and 667 μL of ice, cold isopropyl alcohol was added with gentle mixing to precipitate nucleic acid. A visible, dense clump was seen and centrifuged for ten minutes at 10,000 rpm. The nucleic acid was washed twice with 70% ethanol and allowed to air dry at room temperature followed by further drying with the DNA 110 speed vac (Savant Instruments, Inc.) for five minutes. The pellet was dissolved in 500 μL of 0.1×TE (1 mM Tris-HCl (pH 8)+0.1 mM EDTA (pH 8)) containing 0.1 μg/μL RNase and incubated at 37° C. for thirty minutes. 500 μL of (24:25:1) of chloroform: phenol:isoamyl alcohol was added and mixed thoroughly to visualize three layers and centrifuged for fifteen minutes at 12,000 rpm. The upper layer containing the DNA was removed and placed into new tubes and the same volume of chloroform was added to remove phenol. The tube was mixed thoroughly and centrifuged for fifteen minutes at 14,000 rpm. Two layers were visualized and the top layer containing DNA was transferred to new tubes. A 1 mL volume of 100% chilled ethanol and 33 μL ( 1/10 volume of DNA) of 3M Na Acetate, pH 5.2 were added to allow precipitation of DNA. The reaction was kept at −20° C. for one hour and centrifuged for ten minutes at 14,000 rpm. The supernatant was decanted very slowly with a visible white pellet remaining. The pellet was washed with 70% ethanol and centrifuged for ten minutes at 14,000 rpm. The pellet was dried with a DNA 110 speed vac (Savant Instruments, Inc.) for five minutes to remove remaining ethanol and dissolved in 100 or 200 μL 0.1×TE (1 mM Tris-HCl (pH 8)+0.1 mM EDTA (pH 8)), depending on the pellet size. The genomic samples were loaded on a 0.8% agarose gel to visualize the bands. The concentration of the DNA was determined by a spectrophotometer.

### Restriction Digestion of Genomic DNA

Transgenic and untransformed samples containing equal amounts of DNA were digested with ApaI in a reaction containing: 1.5 μg of DNA, 4 μL of 10×BSA, 4 μL of 10× Buffer 4 (New England Biolabs), 2 μL of ApaI (New England Biolabs), and sterile distilled H20 to make up the volume of 40 μL. The reaction was incubated overnight at 37° C.

### Agarose Electrophoresis and DNA Transfer

Each of the 40 μL reaction volume samples was loaded on a 0.8% agarose gel and electrophoresis was run for 3 hours at 100 volts. The gel was visualized with a fluorescent ruler and was depurinated with 1:40 diluted 12N HCl for ten minutes (until the color of the dye became yellow) and removed. This was followed by immersing the gel into Hybridization Denaturing Solution (5 PRIME-3PRIME) for thirty minutes followed by a wash with sterile distilled water. The gel was then immersed with Hybridization Neutralization Solution (5 PRIME-3 PRIME) for thirty minutes followed by a wash in transfer buffer (2×SSC, Eppendorf) for 5 minutes. The membrane (S & S Nytran: SuperCharge, Schleicher & Schuell) was immersed in sterile distilled water for fifteen minutes and left in transfer buffer until use. The transfer of DNA from the agarose gel to membrane utilized the Turboblotter (Rapid Downward Transfer System/Buffer Tray, Schleicher & Schuell) protocol. Twenty sheets of dry GB004 paper, followed by four sheets of dry GB002, and one sheet of prewet, in transfer buffer, GB002 was placed in the stack tray. The prewet transfer membrane was placed on the stack followed by the agarose gel on top. A rolling pin was used to remove any air bubbles that were present. The top of the gel was wet with the application of transfer buffer and three sheets of prewet GB002 paper was placed on top. The buffer tray was attached to the stack tray and filled with 125 mL of transfer buffer. The wick was placed on top of the stack so the ends would drape into the buffer tray. The wick cover was placed on top to prevent evaporation and the transfer continued overnight at room temperature. The following day, the membrane was marked with a pencil and rinsed with transfer buffer for five minutes. The membrane was placed on chromatography paper and allowed to air dry. The membrane was cross-linked using the UV Stratalinker 2400 (Stratagene) at the setting autocrosslink and stored at a dry place until further use.

### Generation of Probes

The following PCR reaction was set up for the construction of the 2P/2M flanking probe: 1 μL of DNA, 5 μL of 10× Buffer, 2.5 μL of MgCl2, 1 μL of dNTP, 1 μL of 2Pn primer, 1 μL of 2M primer, 1 μL Taq polymerase, and 37.5 μL of sterile H20. The initial denaturation was set at 94° for 5 minutes and amplification was carried out for twenty-nine cycles of the following program: 94° C. for 30 seconds (denaturation), 61° C. for 30 seconds (annealing), and 72° C. for 1 minute and 30 seconds (extension). Final extension of seven minutes at 72° C. was carried at the end of PCR. The 50 μL PCR reaction was loaded into a 0.8% agarose gel and visualized with the Bio Rad Gel Doc 2000. The bands were excised and gel extracted with the QIAquick Gel Extraction Kit (Qiagen) by following the manufacture's protocol and eluting with 35 μL of Buffer EB.

### Prehybridization of Membrane

The membrane was placed in a hybridization bottle with the side exposed to DNA facing inwards and 20 mL of prehybridization solution (36.5 mL of sterile distilled water, 10 mL of 20×SSC, 2.5 mL of 100×Denhardt's (6% Ficoll, 6% polyvinylpyrrolidone, 6% BSA), 500 μL of 10% NaPPi, and 500 μL of 10% SDS) was added and incubated at 65° C. with the Hybridiser HB-1 D (Techne). Sonicated salmon sperm DNA (10 mg/mL) (Stratagene) was boiled for minutes and 500 μL was added into the hybridization bottle. The pre hybridization of the membrane incubated at 65° C. for four hours.

### Probe Labeling and Purification

The PRIME-It Random Primer Labeling Kit (Stratagene) protocol was followed to label the radioactive probe. In a clean microcentrifuge tube: 20 ng of the flanking probe, 10 μL of random oligonucleotide primers, and up to 23 μL of sterile H20 was added and boiled for five minutes. The mixture was briefly centrifuged at room temperature. The following was added in order: 10 μL of 5×ATP buffer, 5 μL of Redivue (Alpha-32P) DATP, and 1 μL of Exo (−) Klenow (5U/μL) and mixed by stirring. The mixture was heated for fifteen minutes at 37° C. followed by adding 2 μL of stop mix. A NucTrap probe purification column (Stratagene) was placed in a push column device and a clean microcentrifuge tube containing 100 μL of salmon sperm DNA was placed below. 80 μL of 1×STE was injected into the column with a syringe and collected into the microcentrifuge tube. The radiolabeled probe was added to the column and pushed through with a syringe followed by 80 μL of 1×STE. The syringe was applied to the column once more and 300 μL of 1×STE was added to the purified probe.

### Hybridization and Washing of Membrane

The purified probe was boiled for five minutes and added to the hybridization bottle with a sterile transfer pipette and allowed to hybridize with the membrane overnight at 65° C. The following day, the membrane was washed twice, five minutes each with wash buffer #1 (2×SSC, 0.1% SDS, and 0.1% NaPPi) followed by washing with wash buffer #2 (0.2×SSC, 0.1% SDS, and 0.1% NaPPi) four times, fifteen minutes each. The wash buffer was discarded into the P32 waste and the membrane was allowed to air dry behind the plastic shield. The radioactive membrane was wrapped with saran wrap and a ladder (Stratagene) was placed on front.

### Autoradiography

The film cassette along with the hybridized blot was taken to the dark room and under safe, red light the blot was placed faced down onto the intensifier screen and the X-ray film was placed in between the screen. The cassette with the hybridized blot and the film was incubated in the dark overnight at −80° C. The next day the cassette was taken out of the −80° C. freezer to thaw and the film was developed with the X-ray film processor.

### Characterization of Expressed Chloroplast-Derived Proteins

Extraction of Protein from Transformed Tobacco Leaves

Mature leaf material was harvested around four to five o'clock in the early evening hours. Leaves were washed in the lab and allowed to air dry and stored at −80° C. until further use. Chloroplast-derived CTB-malarial proteins from transgenic lines was extracted by grinding 100 mg of plant tissue with a mortar and pestle in liquid nitrogen and fine powdered leaf material was placed in a 1.5 mL microcentrifuge tube with a hole poked through the top. The microcentrifuge tube was immediately placed in liquid nitrogen until further use. 200 μL of plant extraction buffer (100 mM NaCl, 10 mM EDTA, 200 mM Tris-HCl pH8, 0.05% Tween 20, 0.1% SDS, 14 mM BME, 200 mM sucrose, 3.18 mL of sterile H20, and 1 tablet of Roche complete mini EDTA-free protease inhibitor cocktail) was added to the leaf material. The samples were placed on ice and mixed for two minutes using a mechanical pestle and centrifuged at 14,000 rpm for fifteen minutes at 4° C. to obtain the supernatant (soluble fraction). The pellet (insoluble fraction) was resuspended with equal volume of protein extraction buffer and sonicated for thirty seconds. The supernatant and pellet was subjected to Bradford analysis to determine total protein concentration and stored at −20° C. until further use.

### SDS-Page and Immunoblot Analysis

Clean Bio Rad glass plates and casting chamber were set up for SDSPage analysis. A 12.5% separating gel (4.15 mL of 30% Bio Rad Acrylamide/Bis solution, 2.5 mL of 4× Separating Buffer: 5M Tris-HCl, pH 8.8, 3.2 mL of H20, 0.1 mL of 10% SDS, 0.1 mL of 10% APS, and 10 μL of TEMED) were mixed in a 50 mL beaker and by using a syringe it was added in between the two glass plates leaving about 1.5 cm empty for the stacking gel. Immediately water was added to the top of the separating gel and allowed to polymerize for thirty minutes. The water was removed with a tissue and 4% stacking gel (665 μL of 30% Bio Rad Acrylamide/Bis solution, 1.25 mL of 4× Stacking Buffer: 0.5M Tris-HCl, pH 6.8, 3.0 mL of H20, 50 μL of 10% SDS, 50 μL of 10% APS, and 5 μL of TEMED) was prepared. The 4% stacking gel mixture was layered on top of the resolving gel and a comb was inserted for the formation of wells. After polymerization for thirty minutes, the gel was put vertically into the PAGE apparatus with 1× Protein Buffer (10× Protein Buffer: 0.25M Tris Base, 1.92M Glycine, and 1% SDS). The protein samples (wild type plants, transgenic plants, and E. coli-derived CTB MSP-1) were prepared by the following: 12 μL of protein extract and 12 μL of 2× gel loading buffer (2.5 mL of 4× Stacking Buffer, 4 mL of 10% SDS, 2 mL of Glycerol, 40 μL of 5% Bromophenol Blue, 0.31 g of DTT in a total of 10 mL of distilled H20). The protein samples were boiled for 5 minutes and loaded into the wells along with 7 μL of Bio Rad Precision Plus Protein Standard. The gel was run at 85 Volts until the protein samples entered into the Separating Buffer and the voltage was increased to 150 Volts until the dye front reached the bottom of the gel. The proteins from the SDS-PAGE were transferred overnight at 4° C. to a HyBond nitrocellulose membrane via the Bio Rad Transfer Cassette using Transfer Buffer (200 mL of methanol, 100 mL of 10× Protein Buffer, and 700 mL of H20) and 20 Volts.

For immunoblot analysis, the membrane was washed three times for five minutes each with PBS-T and blocked for one hour in 5% PTM at room temperature. Primary antibody, Sigma Anti-Cholera Toxin produced in rabbit, was diluted 1:4,000 in 5% PTM and incubated at room temperature for two hours. The membrane was washed three times for ten minutes each with 5% PTM. Secondary antibody, PIERCE Stabilized Goat Anti-Rabbit Horseradish Peroxidase-Conjugated, was diluted 1:5,000 in 5% PTM and incubated at room temperature for one hour. Blots were washed with PBS-T two times for ten minutes each and a final wash of PBS for ten minutes. The membrane was incubated for five minutes in the dark using PIERCE SuperSignal West Femto Maximum Sensitivity Substrate. The membrane was exposed to MIDSCI Classic Blue Autoradiography Film in the dark room and the films were developed via the film processor to visualize the bands.

### Quantification of Expressed Proteins

ELISA was performed to quantify CTB-FC-AMA1 and CTB-MSP-1 in plant crude extract. Transgenic leaf samples of mature stages along with wild type were quantified. Total soluble protein was extracted using the protocol from the section, extraction of protein from transformed tobacco leaves (p.). CTB (Sigma C9903) was used as the standard and diluted in coating buffer (1.59 g of Na2CO3, 2.93 g of NaHCO3, and 0.2 g of NaN3 in 1 L of water; adjusted to pH 9.6 using HCl) ranging from 750-25 pg. Total soluble protein extracted from wild type non-transgenic plants, CTB-FC-AMA-1 plants, and CTB-MSP-1 plants was diluted 1:10, from 1:50,000-1:200,000, and from 1:25,000-1:150,000 in coating buffer, respectively. A 96-well plate (CoStar EIA/RIA plate, flat bottom, without lid, ELISA plate) was coated with 100 μL of CTB standards and test samples and incubated overnight at 4° C. The next day, the plate was washed three times with PBS-T and three times with water. The plate was blocked with 300 μL of 3% PTM and incubated for one hour at 37° C. The plate was washed and 100 μL of primary antibody, Sigma Anti-Cholera Toxin produced in rabbits, was diluted 1:4,000 in 3% PTM and incubated for one hour at 37° C. Following primary antibody, the plate was washed and secondary antibody, Horseradish Peroxidase-Conjugated Donkey Anti-Rabbit (BioMedia), was diluted 1:12,500 in 3% PTM and incubated for one hour at 37° C. The plate was washed and 100 μL of the substrate, TMB (American Qualex Antibodies), was added and incubated at room temperature for 5 minutes. The reaction was stopped with 50 μL of 2N sulfuric acid and the plates were read at 450 nm with the BioRad microplate reader, Model 680. A Bradford assay using the Bradford reagent (Bio-Rad Protein Assay), BSA standards ranging from 0-8 μg/μL, absorbance of 595 nm, and the Bio-Rad SmartSpecPlus Spectrophotometer was used to determine total soluble protein extracted from the wild type and transgenic plants.

To determine the quantity of chloroplast-derived CTB-FC-AMA1 and CTBMSP1 the following equation was used. The concentration (pg) derived from the ELISA multiplied by dilution factor multiplied by 100,000 resulted in concentration of transgenic protein in μg. The concentration of transgenic protein was then divided by the volume of sample (100 μL) placed in the well of the ELISA plate. The number derived after dividing by the volume plated then was divided by the concentration of total soluble protein (provided by Bradford Analysis) and multiplied by 100. The calculated percentage provides an estimate of the chloroplast-derived protein accumulation among all proteins expressed by the plant.

### Enrichment of Chloroplast-Derived Proteins

### Talon Purification

Chloroplast-derived CTB-malarial proteins from transgenic lines was extracted by grinding 10 g of plant tissue with a mortar and pestle in liquid nitrogen and fine powdered leaf material was placed in a 50 mL conical tube with a hole poked through the top. The conical tube was immediately placed in liquid nitrogen until further use. 20 mL of plant extraction buffer (100 mM NaCl, 200 mM Tris-HCl pH8, 0.05% Tween 20, 0.1% SDS, 200 mM sucrose, 12 mL of sterile H20, and 1 tablet of Roche complete mini EDTA-free protease inhibitor cocktail) was added to the leaf material. The samples were placed on ice and homogenized for five minutes with an OMNI International (GLH-2596) probe and centrifuged at 14,000 rpm for fifteen minutes at 4° C. to obtain the supernatant (soluble fraction).

The supernatant (lysate) was subjected to TALON Superflow Metal Affinity Resin (Clontech) to enrich the chloroplast-derived CTB-malarial proteins. The manufacture's protocol, BATCH/Gravity-Flow Column Purification, was followed exactly. The TALON Resin was resuspended thoroughly and 4 mL was placed in a sterile 50 mL conical tube and centrifuged at 700×g for two minutes to pellet the resin. The pellet was pre-equilibrated twice with ten bed volumes of 1× Wash Buffer (2.5 mL of 4× Wash Buffer: 0.12M dibasic Na2HPO4, 0.08M monobasic NaH2PO4, 1.2M NaCl, 4% Tween-20, made up to 100 mL of sterile H20, pH8; 20 mM Imidazole, sterile H20 was added to make up the volume to 10 mL, and 1 tablet of Roche complete mini EDTA-free protease inhibitor cocktail). The plant extract was added to the resin and agitated at 4° C. for two hours. The mixture was centrifuged at 700×g for five minutes and the supernatant (flow through) was removed carefully without disturbing the resin. The mixture was washed twice with ten bed volumes of 1× Wash Buffer and the supernatant was discarded. 2 mL of 1× Wash Buffer was added to the resin and transferred to a 2 mL gravity-flow column with an end-cap in place. The end cap was removed and the buffer was allowed to drain. The column was washed once with five bed volumes of 1× Wash Buffer. To elute the chloroplast-derived CTB-malarial proteins, five bed volumes of Elution Buffer (2.5 mL of 4× Wash Buffer, 20 mM Imidazole, 100 mM EDTA, volume made up to 10 mL of sterile H20, and 1 tablet of Roche complete mini EDTA-free protease inhibitor cocktail, pH8) was added and the eluate was collected. The eluted fraction collected along with wild type material, lysate, flow through, and wash was analyzed with a Bradford and the Bio-Rad RC-DC Protein Assay to determine protein concentration. The eluted fractions were dialyzed with 1× sterile PBS and the Slide-A-Lyzer Dialysis Cassette 10,000 MW (PIERCE).

### Analysis of Talon Purification

The eluted, wild type material, lysate, flow through, and wash fractions were subjected to a gradient gel and immunoblot to determine the efficiency of enrichment. 7 μg of the CTB FC AMA-1 fractions under reduced and non-reduced conditions were heated at 70° C. for ten minutes and loaded into a NuPAGE Novex Bis Tris Gel (Invitrogen) and electrophoresed at 200 Volts until the dye front reached the bottom of the gel. The gel was rinsed in water and stained overnight with the GelCode Blue Stain Reagent (PIERCE). A 5 μg sample of the CTB FC AMA-1 and CTB MSP-1 fractions (eluted, wild type, lysate, flow through, wash) were electrophoresed and analyzed by an immunoblot. The primary antibody, Sigma Anti-Cholera Toxin produced in rabbit, diluted 1:4,000 in 5% PTM, was incubated at room temperature for two hours. Secondary antibody, PIERCE Stabilized Goat Anti-Rabbit Horseradish Peroxidase-Conjugated, diluted 1:5,000 in 5% PTM, was incubated at room temperature for one hour. Following incubation with substrate, membranes were exposed to X-ray film and developed via the film processor to visualize the bands.

### Densitometry

An immunoblot of the eluted chloroplast-derived CTB-malarial proteins and known quantities of CTB protein was analyzed by using spot densitometric analysis. 1000, 500, 250, 125 ng of CTB protein (Sigma, C9903) and 1.5, 0.75, 0.375, 0.1875 μg of eluted CTB FC AMA-1 and 1.5, 0.75, 0.375 μg of eluted CTB MSP-1 was electrophoresed and analyzed by an immunoblot. The primary antibody added was rabbit anti-CTB and the secondary antibody was goat anti-rabbit. Following exposure to film, the blots with known CTB concentrations and eluted fractions were analyzed by using the AlphaImager and AlphaEase FC Software (Alpha Innotech). The concentration of the enriched fraction the program calculated was divided by the known concentration of the enriched fraction loaded and multiplied by one hundred to determine efficiency of the talon enrichment.

### Immunization Studies in Mice

### Adsorption of Protein to Adjuvant

Chloroplast-enriched proteins (˜2.5 mg) derived from transgenic tobacco crude extract were mixed with 1:4 diluted Alhydrogel in PBS (Aluminum Hydroxide Gel, Sigma) and incubated overnight with gentle rocking at 4° C. The samples were centrifuged at 2,000×g for five minutes at 4° C. The Bio-Rad RC-DC Protein Assay was used to determine the adsorption efficiency by comparing the total amount of protein added to the adjuvant and the protein remaining in the supernatant after binding to adjuvant. The protein-adsorbed pellet was resuspended in sterile PBS to a final concentration of 1 μg/μL.

### Immunizations

For subcutaneous injections 100 μL of Alhydrogel or chloroplast-derived protein adsorbed to Alhydrogel was injected into the scruff of the neck using a tuberculin syringe fitted with a 27-G needle. The leaf material for the doses for oral delivery was previously ground in liquid nitrogen with mortars and pestles and stored at −80° C. until the day of immunization. Oral doses (500 mg each) of either wild type or transgenic leaf material were resuspended in 200 μL of sterile PBS and homogenized on ice for five minutes with an OMNI International (GLH2596) probe. The plant cell suspension was stored on ice until oral delivery. A 200 μL aliquot of the plant cell suspension was delivered via oral gavage by using a tuberculin syringe and a 20-G bulb-tipped gastric gavage needle.

Ninety female BALB/c mice were purchased at 7 weeks from Charles River Laboratories and were immunized by the following schedule listed below, in Table 1 (next page).

Determination of Antibody Titers from Serum Samples

### Blood Sample Collection

Blood samples were obtained on days 21, 35, 63, 163, and 197-post immunization. The mouse was restrained and blood was collected by inserting a golden rod animal lancet 4 mm, 5 mm, or 5.5 mm, depending on the age and size of the mouse, in the submandibular vein. The blood was collected in Microtainer serum separation tubes (Becton-Dickinson) and allowed to clot for a minimum of 30 minutes at room temperature. The blood samples were centrifuged at 15,000 rpm for 5 minutes and the serum was transferred to new microcentrifuge tubes and stored at −80° C.

### ELISA to Determine Antibody Titers

Several 96-well plates (CoStar EIA/RIA plate, flat bottom without lid ELISA plate) were coated with 50 ng of MRA-56 PfMSP1₁₉ protein in coating buffer (1.59 g of Na₂CO₃, 2.93 g of NaHCO₃, and 0.2 g of NaN₃ in 1 L of water; adjusted to pH 9.6 using HCl) per well. The plates were incubated overnight at 4° C. The following day, the plates were washed three times with PBS-T and water and blocked with PBS containing 0.1% Tween and 3% skim milk powder for one hour at 37° C. The serum samples from groups 5, 6, and 9 (unimmunized) were diluted in PTM with the following dilutions: bleed #1 and #2 ranging from 1:100 to 1:1000; bleed #3 from 1:100 to 1:10,000; bleed #4 from 1:500 to 1:75,000; and bleed #5 from 1:1000 to 1:100,000. The plates were washed and incubated with 100 μL of diluted serum samples (in duplicate) and incubated for one hour at 37° C. The plates were washed and incubated with 100 μL of 1:5,000 diluted goat anti-mouse IgG1 (American Qualex) conjugated with horseradish peroxidase enzyme in PTM for one hour at 37° C. The plates were washed and 100 μL of TMB substrate (American Qualex Antibodies) was added and incubated at 37° C. for thirty minutes. The reaction was stopped with 50 μL of 2N sulfuric acid and the plates were read at 450 nm with the plate reader (BioRad microplate reader, Model 680). The titer values were determined by using the O.D. (optical density) of the negative control (unimmunized mice, group 9)+0.1.

### Immunoblot Detection of Anti-Malarial Antibodies In Serum Samples

The protocol followed was mentioned above in the characterization of chloroplast-derived proteins (p.). The protein samples loaded into the SDSPAGE gel were isolated prior and stored in the −80° freezer. Samples of 36.8 μg of ring, trophozoite, and schizont stage proteins were electrophoresed and analyzed by an immunoblot. The primary antibody, sera collected from an immunized mouse from groups 3 and 5, used was diluted 1:100 in 5% PTM. Secondary antibody, PIERCE Stabilized Goat Anti-Mouse Horseradish Peroxidase-Conjugated, was diluted 1:5,000 in 5% PTM. Following incubation with substrate, membranes were exposed to X-ray film and developed via the film processor to visualize the bands.

### IFA Detection of Anti-Malarial Antibodies in Serum Samples

A 3D7 P. falciparum culture was resuspended and centrifuged to collect the pellet for examination of the RBCs containing the parasites. A revised protocol by Tonkin et al. was followed for the preparation and fixation of cells (Tonkin, van Dooren et al. 2004). Cells were washed once with PBS and fixed in 4% paraformaldehyde and 0.0075% glutaraldehyde in PBS for 30 min at room temperature. Following fixation, cells were washed once with PBS and permeabilized with 0.1% Triton X-100 (Sigma) for ten minutes and to reduce free aldehydes cells were treated with 0.1 mg/mL NaBH4/PBS for ten minutes at room temperature. Cells were washed once with PBS and finally blocked with 3% BSA/PBS for one hour at room temperature. Cells were probed with antibodies, (sera obtained from an immunized mouse in group 3 and 5) diluted 1:500 in 3% BSA/PBS for two hours at room temperature followed by three washes with PBS. The secondary antibodies were Alexa Fluor 555 goat anti-mouse diluted 1:1000 in 3% PBS containing BSA and inverted in the dark for one hour at room temperature. Cells were washed three times with PBS and allowed to settle on previously coated coverslips with 1% PEI for thirty minutes at room temperature. The mounting solution, 50% glycerol with 0.1 mg/mL DABCO (Sigma) was added to the coverslips and then inverted on microscope slides. Fluorescence images were observed and captured by using an LSM 510 confocal laser scanning microscope (Carl Zeiss).

In vitro Parasite Inhibition Assay

The 3D7 P. falciparum culture was synchronized with ring stage parasites and sorbitol lysis. The parasite completed one cycle and was allowed to mature to the trophozoite-schizont stage. The hematocrit and parasitemia were adjusted to 2%. Mouse sera from the nine groups and MRA-35 PfMSP1₁₉ (positive control) were heat inactivated at 56° C. for 30 minutes and absorbed with human RBCs overnight at 4° C. The mouse serum was added to the parasite culture in 96 well plates at a final concentration of 20%. To serve as a negative control, no serum was added to wells and replaced with culture media. The cultures were incubated for 48 hours to allow for schizont rupture and merozoite invasion. These assays were preformed in duplicate. For microscopic analysis using the 100× oil immersion lens, blood smears were made and stained with Giemsa and the numbers of ring stage parasites per 900 to 1,100 RBCs were determined for each well. Parasitemia was measured using the following formula (Infected RBCs/infected+uninfected RBCs)×100. Photomicrographs were taken from the slides to show the extent of parasitemia and inhibition under light microscopy.

### Results

### PCR Analysis of CTB, AMA-1, and MSP-1 Amplification

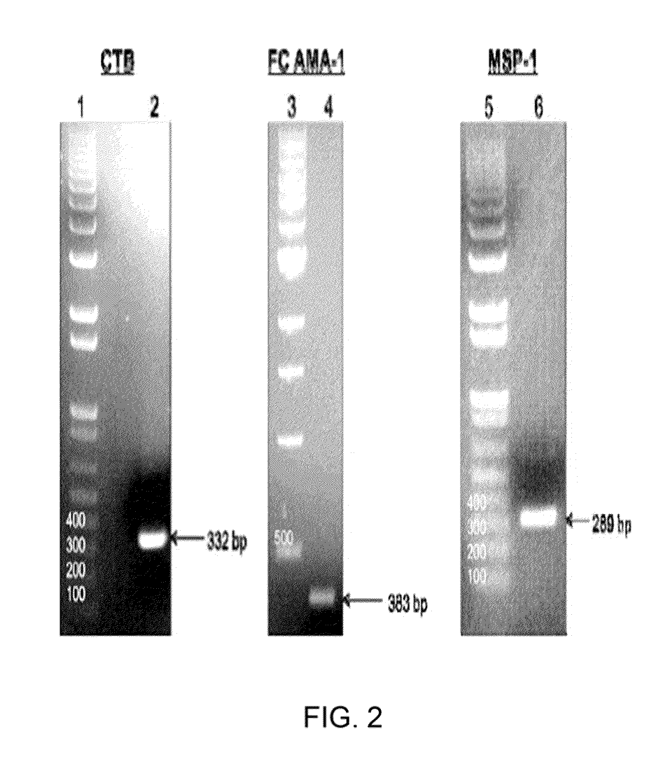

To verify AMAL and MSP-1 expression in 3D7 P. falciparum, transcripts were detected by RT-PCR by using total RNA from mixed stages of parasites and gene-specific primers (data not shown). By using gene-specific primers, template DNA, and PCR: FC AMA-1, MSP-1, and CTB were amplified with the expected sizes as depicted in FIG. 2: PCR Analysis of CTB, FC AMA-1, and MSP-1.

DNA was amplified using a single tube PCR approach and gene specific primers as discussed under “Materials and Methods” resulting in Lane 2: (CTB, 332 bp), Lane 4: (FC AMA-1, 383 bp), and Lane 6: (MSP-1, 289 bp). Molecular size standards are indicated in Lanes 1, 5: 1 kb+ladder and Lane 3:1 kb ladder.

Cloning Analysis into Chloroplast Vector

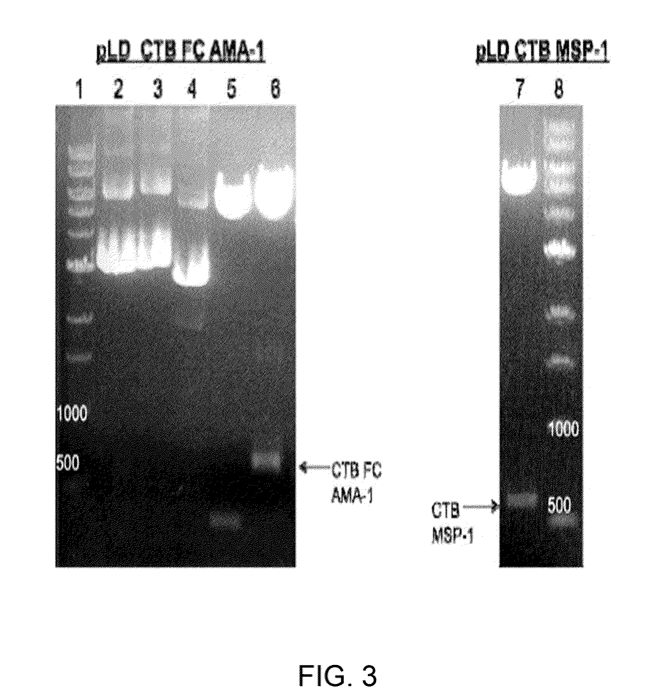

The genes FC AMA-1, MSP-1, and CTB were successfully cloned into the pGMET easy and pBSK+ vectors successfully (data not shown). The DNA sequences were confirmed after successful ligation into the pGEMT easy vector. Once the transgene fusions, CTB FC AMA-1 and CTB MSP-1, were constructed in the pBSK+ vector, they were excised and ligated into the chloroplast pLD-UTR vector with expected sizes as shown in FIG. 3. The constructs were then bombarded into the tobacco plant, Nicotiana tabacum variety Petit Havana, by the protocol listed in the “Materials and Methods” section.

FIG. 3 shows Analysis of Cloning CTB FC AMA-1 and CTB MSP-1 Into the pLD-UTR Chloroplast Vector. After purifying plasmid DNA with the Qiagen Plasmid Maxi Kit, the DNA was analyzed by gel electrophoresis. CTB FC AMA-1 is depicted in Lane 3: undigested and Lane 6: digested with NotI and NdeI resulting in a 715 bp fragment. CTB MSP-1 is illustrated in Lane 7: after digestion with NotI and NdeI (621 bp fragment). In Lanes 2, 4, 5: genes were inserted into the pLD-UTR vector but were not further studied. Molecular size standards are indicated in Lanes 1, 8: 1 kb ladder.

### Confirmation of Chloroplast Integration of Transgenes

The gene cassette (aadA and CTB FC AMA-1/CTB MSP-1) was introduced into the tobacco chloroplast genome through homologous recombination of the flanking sequences of the pLD-UTR vector (trnI and trnA) and the native plastid genome. The Prrn downstream of the trnI gene is for transcription of aada that confers resistance to spectinomycin and streptomycin and the transgene that encodes the CTB-malarial gene. The 3′ UTR upstream of the trnA gene provides stability for the transcript and may be involved in ribosome recruitment.

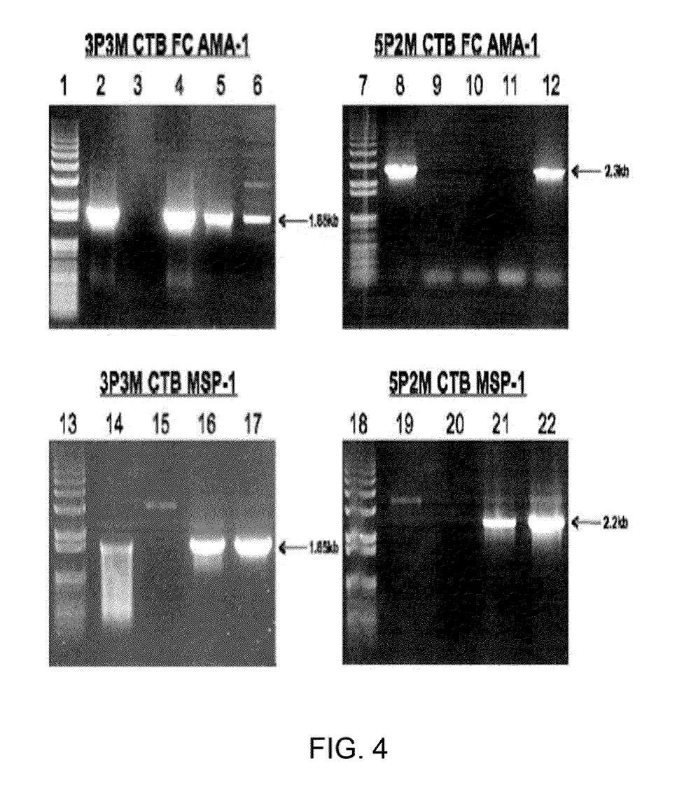

Resistant shoots appeared on the regeneration media (RMOP) containing spectinomycin. Using specific primers and PCR analysis, mutant and nuclear integrated shoots were differentiated from shoots where the transgene integrated into the chloroplast genome. By using 3P/3M primers, site-specific integration of the gene cassette was confirmed. To eliminate all nuclear transformants, the 3P primer was used because it annealed to the native chloroplast genome. The 3M primer was used because it landed on the aadA gene and eliminated all mutant transformants. The 3P/3M primers, resistant shoots, and PCR analysis yielded a 1.65 kb band as shown in FIG. 4 (left panels). Another set of primers 5P/2M were used to confirm the integration of the aadA gene and the CTB-malarial gene. The 5P primer annealed to the aadA gene and the 2M primer annealed to the trnA gene. The amplified PCR product size was 2.3 kb for CTB FC AMA-1 and 2.2 kb for CTB MSP-1 as shown in FIG. 4 (right panels). The 5P/2M PCR analysis eliminated all mutants and only positive transformants with transgene integration were visualized. The positive transformants were subjected to second and third round of selection to confirm homoplasmy.

FIG. 4 depicts the PCR Analysis of Wild Type and Positive Transformants. PCR using specific primers land within the native chloroplast genome and the aadA gene (3P3M) to yield a 1.65 kb product Lanes 2, 14: positive control, Lanes 3, 15: wild type, Lanes 4-6: transgenic lines pLD-UTR CTB FC AMA-1, and Lanes 16, 17: transgenic lines pLD-UTR CTB-MSP-1. Using the specific 5P/2M primers, landing on the aadA gene and tmA gene, respectively yields a 2.3 kb and 2.2 kb product Lanes 8, 19: positive control, Lanes 9, 20: wild type, Lanes 10, 11: negative transformants pLD-UTR CTB FC AMA-1, Lane 12: transgenic line pLDUTR CTB FC AMA-1, and Lanes 21, 22: transgenic lines pLD-UTR CTB-MSP-1. Molecular size standards are indicated in Lanes 1, 7, 13, 18: 1 kb+ladder.

### Southern Analysis of Transgenic Plants

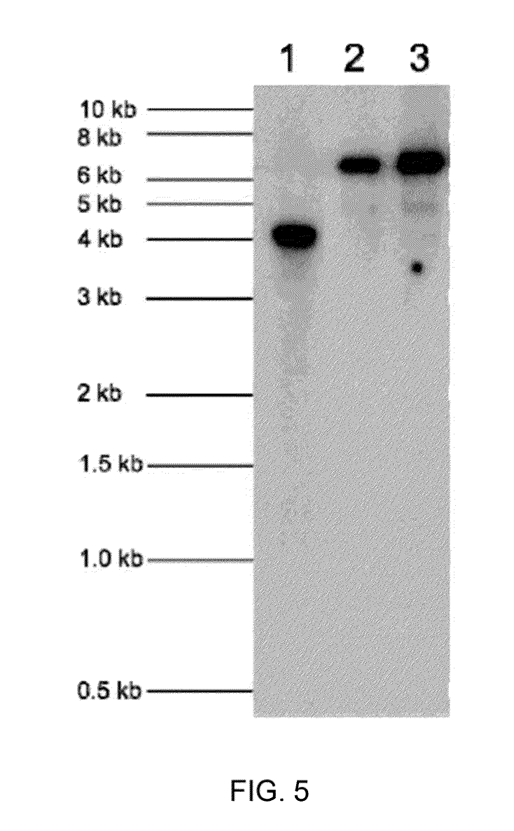

To confirm site-specific integration and homoplasmic plants, transgenic plant genomic DNA was probed with the 2P/2M flanking sequence probe of 1.3 kb in size. The transgenic and non-transgenic genomic DNA was digested with the restriction enzyme ApaI. After digestion, the untransformed chloroplast genome resulted in a 4.5 kb fragment and the transformed chloroplast genome resulted in 6.5 and 6.6 kb fragments for CTB MSP-1 and CTB FC AMA-1, respectively, as depicted in FIG. 5.

FIG. 5 illustrates the Evaluation of Transgene Integration into the Chloroplast Genome of Homoplasmic Plants by Southern Blot. Southern blot probed with the 2P/2M flanking sequence probe to determine homoplasmy Lane 1: wild type, Lane 2: homoplasmic CTB MSP-1 (6.5 kb), and Lane 3: homoplasmic CTB FC AMA-1 (6.6 kb).

### Selection and Generation of Transgenic Plants

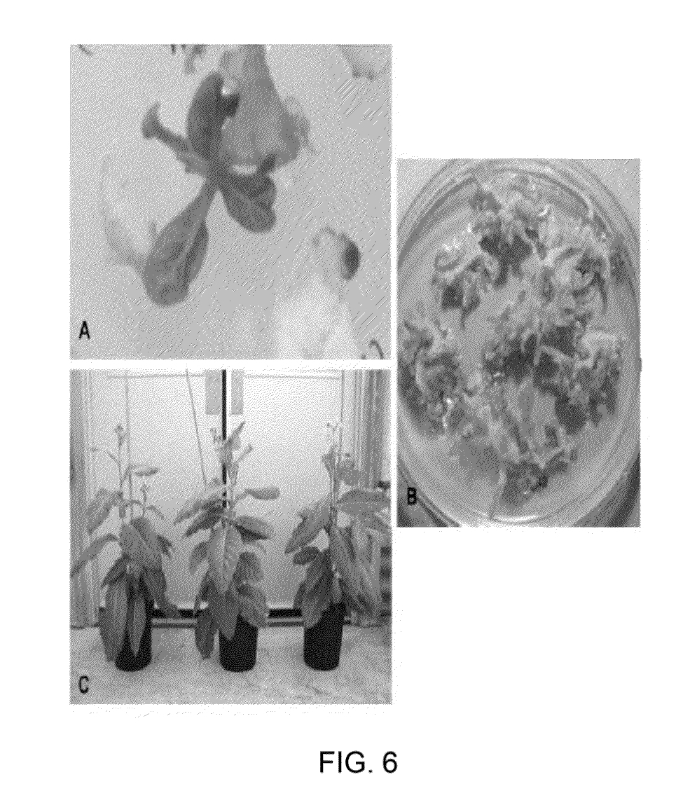

Transgenic plants were selected and generated by the use of RMOP media and spectinomycin (FIG. 6A). After positive transformants were determined by PCR analysis, further selection and generation of transgenic shoots was maintained (FIG. 6B). MSO selection media containing spectinomycin was used for rooting. Once the in vitro plants were confirmed homoplasmic by Southern Blot analysis the plants were transferred to the greenhouse for optimal growth and maximal protein expression (FIG. 6C).

FIG. 6 is captioned Generation of Transgenic Plants. (A) After four to five weeks of particle bombardment, transplastomic shoots appear on RMOP selection medium. For second round of selection, leaves from PCR positive transformants are transferred to RMOP selection medium. (B) Several shoots appear within two to three weeks. For third round of selection, regenerated shoots are transferred to MSO selection medium and roots appear in about 10 days. (C) Plants were transferred to the greenhouse after confirmation of homoplasmic plants via Southern blot analysis.

### Immunoblot Analysis

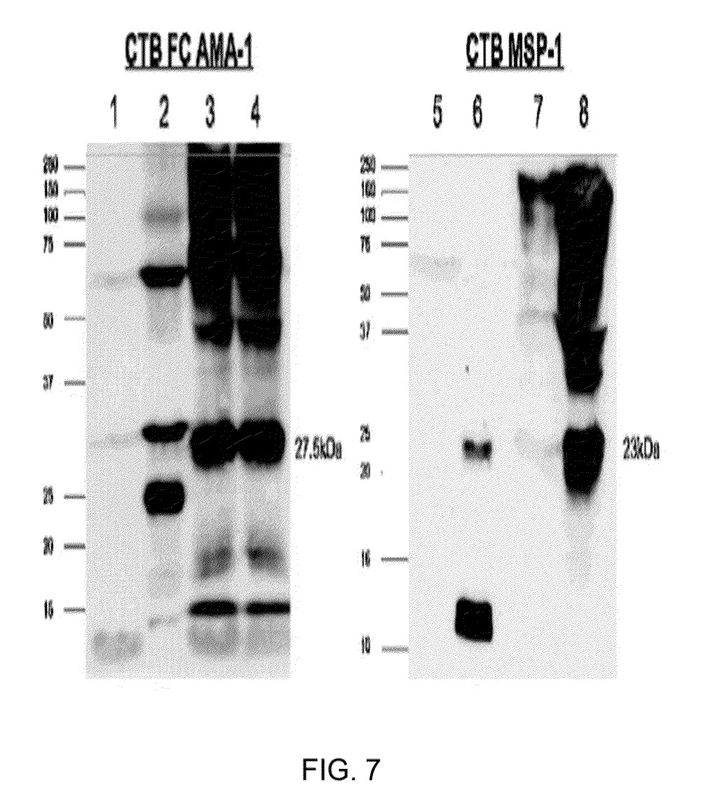

Crude extracts of approximately 50 μg of wild type and transgenic plants along with a E. coli expressed CTB MSP-1 extract were loaded in the wells of a SDS-PAGE gel. The blots were probed with the polyclonal anti-CTB primary antibody and the monomeric forms of CTB FC AMA-1 showed a 27.5 kDa protein and CTB MSP-1 depicted a 23 kDa protein in the insoluble (pellet) and soluble (supernatant) fractions (FIG. 7). The immunoblot displayed others forms of the CTB-malaria proteins such dimers, trimers, tetramers, and pentamers (FIG. 7).

FIG. 7 shows the Immunoblot Analysis to Confirm Expression of CTB-Malaria Antigens in Nicotiana tabacum Crude Extracts. Immunoblot with anti-CTB polyclonal antibody showed full-length protein. Lanes 1, 5: wild type, Lanes 2, 6: positive control CTB MSP-1 E. coli expressed, Lane 3: CTB FC AMA-1 pellet, Lane 4: CTB FC AMA-1 supernatant, Lane 7: CTB MSP-1 pellet, and Lane 8: CTB MSP1 supernatant.

### Protein Quantification Using an ELISA

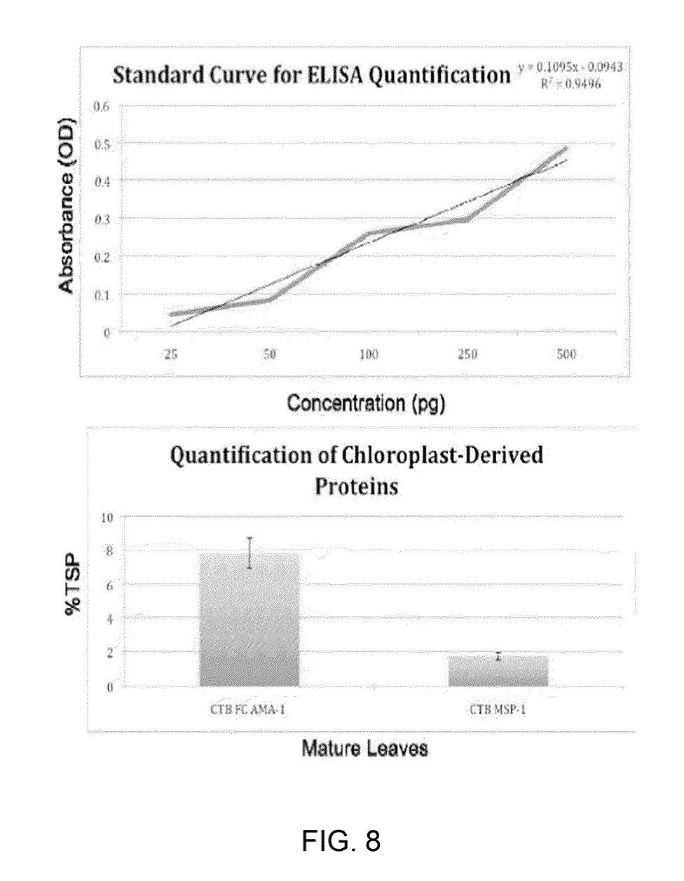

To quantify the protein levels of the chloroplast derived CTB-malarial proteins, CTB protein of known concentrations was optimized as depicted in FIG. 8 (top panel). The CTB FC AMA-1 and CTB MSP-1 protein expression levels of mature leaves reached 6.3-9.5% and 1.4-2%, respectively as shown in FIG. 8 (bottom panel). The accumulation of the CTB-malarial proteins in high level of expression is due to the presence of high number of chloroplasts and chloroplast genomes (up to 10,000 copies per cell).

FIG. 8 indicates the Quantification of Chloroplast-Derived CTB-Malarial Expression. An ELISA with the following expression levels determined quantification: 6.3-9.5% of CTB FC AMA-1 and 1.4-2% of CTB MSP-1 of total soluble protein of mature leaves.

### Enrichment of Chloroplast-Derived CTB-Malarial Proteins

### Resolution of Enriched Proteins

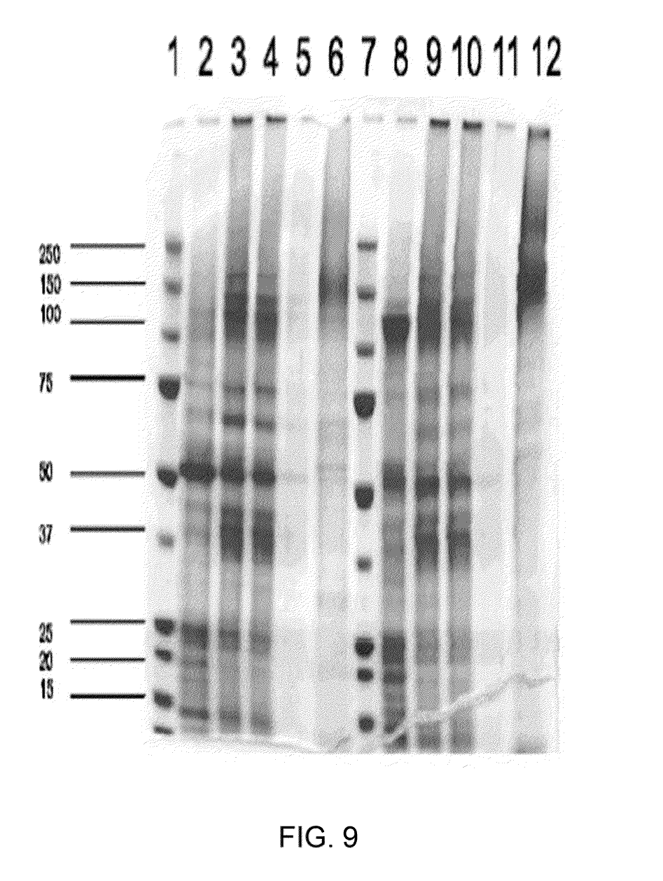

A crude extract of chloroplast-derived proteins was subjected to talon purification and analysis followed. A gradient gel was used to increase the resolution of the enriched CTB FC AMA-1 protein. The gel was performed under reduced and non-reduced conditions. The large subunit of rubisco (55 kDa) is apparent in the wild type, lysate, and flow through fractions under reduced and non-reduced conditions (FIG. 9). In the wash fractions there are minimal bands present. In the eluted CTB FC AMA-1 fraction, the monomer of 27.5 kDa in size is present under reduced conditions and the pentameric form is present under both reduced and non-reduced conditions (FIG. 9).

FIG. 9 shows Increased Resolution of Chloroplast-Derived CTB FC-AMA-1 Protein After Talon Purification. CTB FC-AMA-1 protein was extracted from transformed leaves and the crude extract was subjected to Talon Superflow Metal Affinity Resin and analyzed. Lanes 2-6: reduced and Lanes 8-12: non-reduced conditions of CTB FC-AMA-1 protein enrichment was observed by using a gradient gel (4-12%) and gel electrophoresis. The following fragments were visualized Lanes 2, 8: wild type, Lanes 3, 9: lysate, Lanes 4, 10: flow through, Lanes 5, 11: wash, and Lanes 6, 12: enriched protein. Molecular size standards are indicated in Lanes 1, 7: 1 kb ladder.

### Immunoblot Analysis of Enriched Proteins

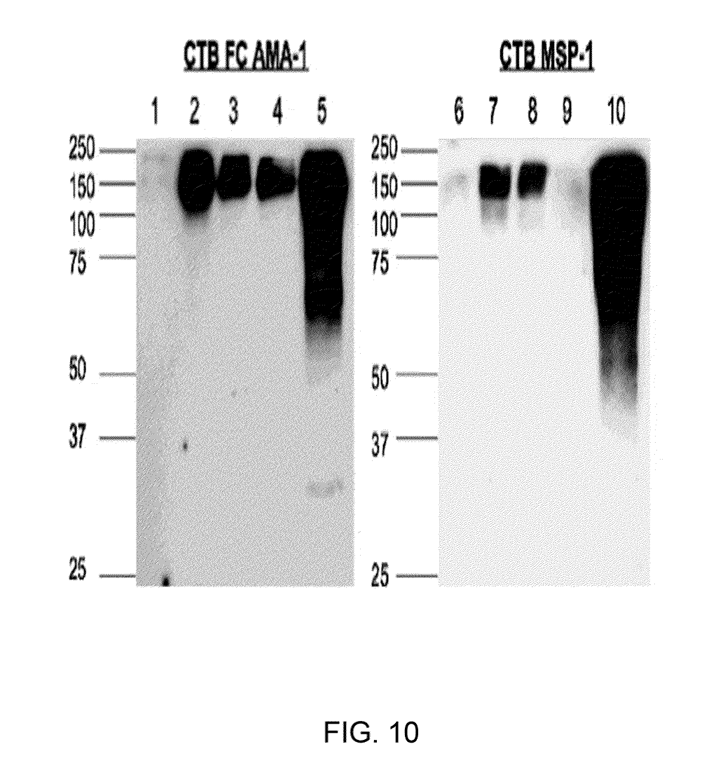

An immunoblot probed with anti-CTB antibody was conducted to confirm the presence of the CTB-malarial proteins after talon enrichment. Equal amounts of protein from wild type, lysate, flow through, wash, and enrichment was loaded into the gel and visualized via an immunoblot. In the fractions containing protein harvested from wild type leaves resulted in no apparent band in the immunoblot analysis (FIG. 10). Lower levels of CTB-malarial proteins were found in the flow through and wash fractions compared to the lysate and in the enriched fractions significant protein expression were found confirming enrichment of the CTB-malarial proteins.

FIG. 10: Immunoblot Analysis of Enrichment of Malarial Antigens from Nicotiana tabacum Extracts. Malaria protein was extracted from transformed leaves and the crude extract was subjected to Talon Superflow Metal Affinity Resin and the following fractions were collected and analyzed using an immunoblot with polyclonal anti-CTB antibody: Lanes 1, 6: wild type, Lanes 2, 7: lysate, Lanes 3, 8: flow through, Lanes 4, 9: wash, and Lanes 5, 10: eluted CTB FC AMA-1 and CTB MSP-1, respectively.

### Densitometric Analysis

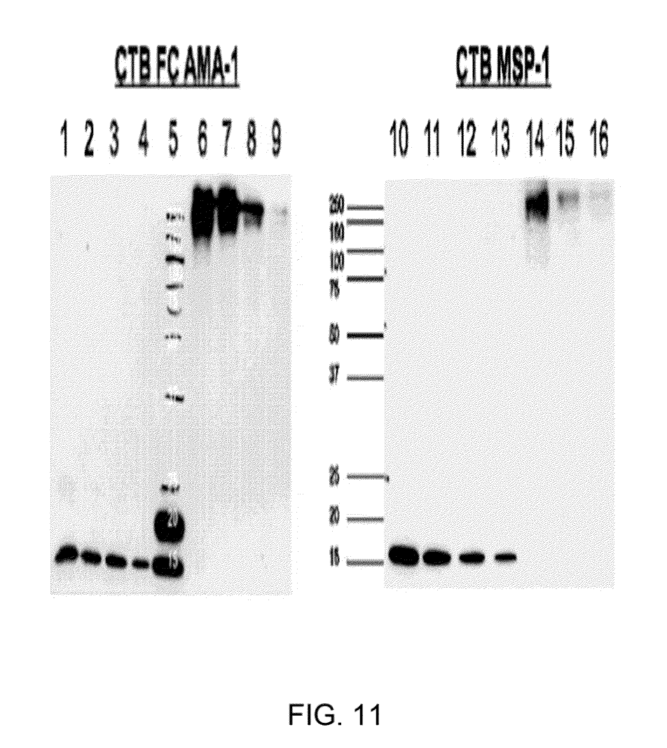

An immunoblot with known concentrations of CTB protein and different concentrations of the enriched fractions were probed with anti-CTB antibody. Quantification of the enriched CTB-malarial proteins on immunoblots was analyzed by spot densitometry. Linearity of the standard curve was achieved by using 1000, 500, 250, and 125 ng of CTB (data not shown) and assisted in the estimation of the enriched samples in the same blot (FIG. 11). The standard curve provided the concentration of the enriched fractions and the efficiency of the talon enrichment was determined to be 90% and 73% in CTB FC AMA-1 and CTB MSP-1, respectively.

FIG. 11: Immunoblot of the Eluted Protein Fractions were Analyzed and Compared to Known Quantities of CTB Protein. Immunoblot analysis of Lanes 1-4 and 10-13: CTB protein (1000, 500, 250, 125 ng, respectively), Lanes 6-9: eluted CTB FC AMA-1 (1.5, 0.75, 0.375, 0.1875 μg, respectively), and Lanes 14-16: eluted CTB MSP-1 (1.5, 0.75, 0.375 μg, respectively). Eluted proteins and CTB were subjected to densitometry to determine the enrichment of CTB FC AMA-1 and CTB MSP-1 to be administered to mice for subcutaneous injection.

### Immunization of Mice

The sera collected from the five bleeds following immunization were on days 21, 35, 63, 163, and 197-post immunization. Each serum was tested for anti-PfMSP1₁₉ antibodies by a capture ELISA with MRA-56 PfMSP1₁₉ protein. Minimal mouse titers were detected from bleeds #1 and #2 and titers were present ranging from 1:100-1:50,000 in bleeds #3, #4, and #5 (Table 2). Titers for anti-PfMSP1₁₉ were found to be higher in group 5 (subcutaneous injection) than in group 6 (oral delivery) (Table 2). One mouse in group 5 (5B3) and three mice in group 6 (6A1, 6A3, 6B4) depicted undetectable titers with MRA-56 PfMSP1₁₉ protein (Table 2; next page).

### Recognition of Native Parasite Protein by Anti-Malarial Antibodies

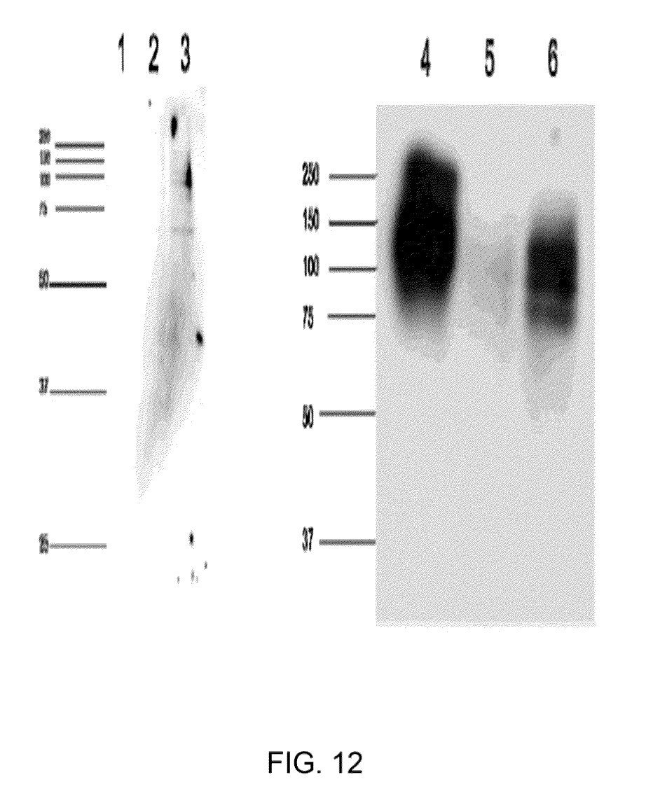

An immunoblot confirmed anti-malarial antibodies from immunized mice recognized native parasite protein from P. falciparum parasite culture. Anti-AMA1 antibodies recognized the schizont stage with the presence of an 83 kDa band and other proteolytically processed fragments. The serum from an immunized mouse from group 5 contained anti-MSP-1 antibodies, which recognized ring and schizont stages with an apparent 190 kDa band (FIG. 12).

FIG. 12: Recognition of Native Parasite Protein by Anti-AMA-1 and Anti-MSP1 Antibodies. Immunoblot displaying Lanes 1-3: anti-AMA-1 collected from an immunized mouse recognizes native 83 kDa AMA-1 protein and proteolytically processed fragments and Lanes 4-6: anti-MSP-1 recognizes 190 kDa MSP-1 protein. The parasite stages analyzed from the 3D7 P. falciparum culture include Lanes 1, 4: ring, Lanes 2, 5: trophozoite, and Lanes 3, 6: schizont.

### Recognition of Native Parasite by Anti-Malarial Antibodies

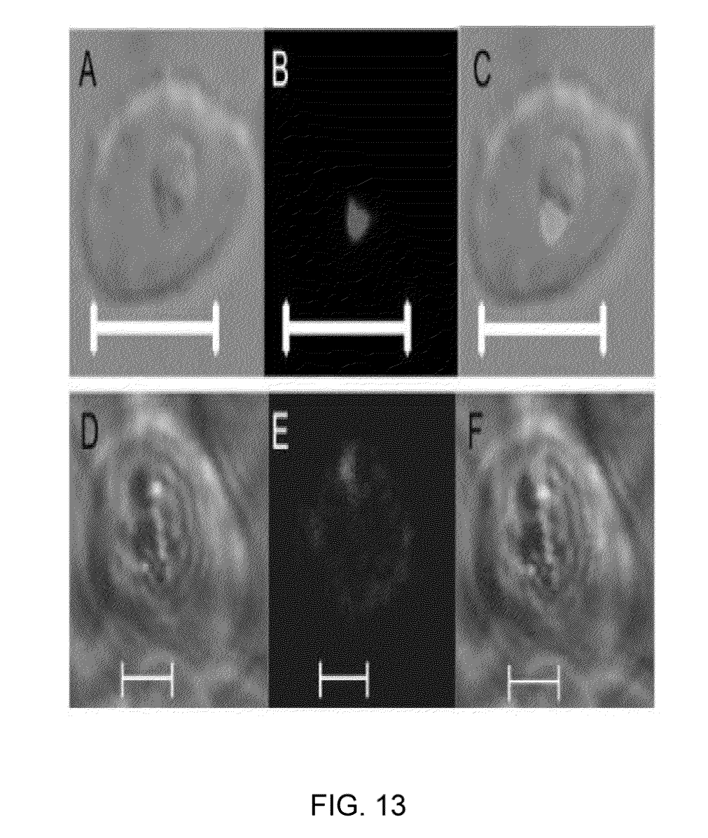

Sera collected from immunized mice with chloroplast-derived CTB-malarial antigens resulted in the recognition of native parasite. Anti-AMA-1 antibodies were found in the immunized sera because native parasites were stained in the apical end of the parasite (FIG. 13 B, C). A mouse immunized with the chloroplast-derived CTB-MSP-1 antigen resulted in stained schizonts indicating the presence of anti-MSP-1 antibodies (FIG. 13 E, F).

FIG. 13: Recognition of Native Parasite by Anti-AMA-1 and Anti-MSP-1 Antibodies. Visible and immunofluorescence (IFA) images of 3D7 P. falciparum parasite immunostained with (A, B, C) anti-AMA-1 raised in mice displaying recognition of the apical end of the parasite and (D, E, F) anti-MSP-1 collected from immunized mouse sera recognizing the MSP-1 of a schizont parasite.

### In Vitro Parasite Inhibition Assay

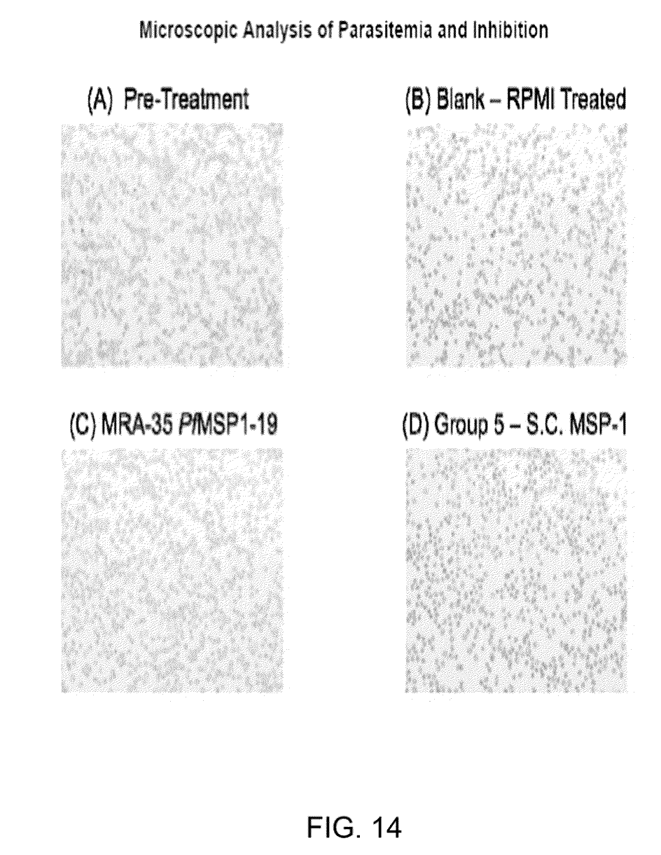

An in vitro parasite inhibition assay was performed to test the ability of anti-AMA-1 and MSP-1 antibodies in inhibiting parasite entry into red blood cells. Synchronized trophozoite-schizont stage parasites (2% parasitemia and 2% hematocrit, FIG. 14 A) were incubated with control and test sera for forty-eight hours and blood smears were made. The slides were stained with Giemsa and the number of parasites and total number of RBCs were counted. The parasitemia was determined and the percent inhibition was calculated. The predominant stage found under microscopic examination was the ring stage. The average parasitemia for the blank control (no serum added) was determined to be 6.6% (FIG. 14 B) while the lowest parasitemia found was from group 5 (2.5%, FIG. 14 D) representing the group with highest percent in inhibition of parasite invasion (Table 3). The serum from the positive control (MRA-35 rabbit antiserum against purified recombinant yeast secreted PfMSP1₁₉, 3D7, FIG. 14 C) resulted in only 53% inhibition (Table 3). The control groups (groups 1, 2, 9) resulted in 7.6-13.6% inhibition and the remaining experimental groups (groups 3, 4, 6, 7, 8) resulted in 45.5-56.1% inhibition (Table 3; next page).

### Microscopic Analysis of Parasitemia and Inhibition

FIG. 14: Microscopic Examination of the in vitro Parasite Inhibition Assay. Synchronized 3D7 P. falciparum trophozoite-schizont stage culture (2% parasitemia and hematocrit, FIG. 14 A) and no sera (FIG. 14 B), MRA-35 PfMSP1₁₉ sera (FIG. 14 C), and immunized mouse sera from group 5 (FIG. 14 D) was incubated for 48 hours; the parasitemia was estimated by counting infected and uninfected RBCs.

### The Expanded Study With Edible Plants

### Expression of CTB-AMA1 and CTB-MSP1 in Lettuce Chloroplasts

### Chloroplast Transformation Vector Construction

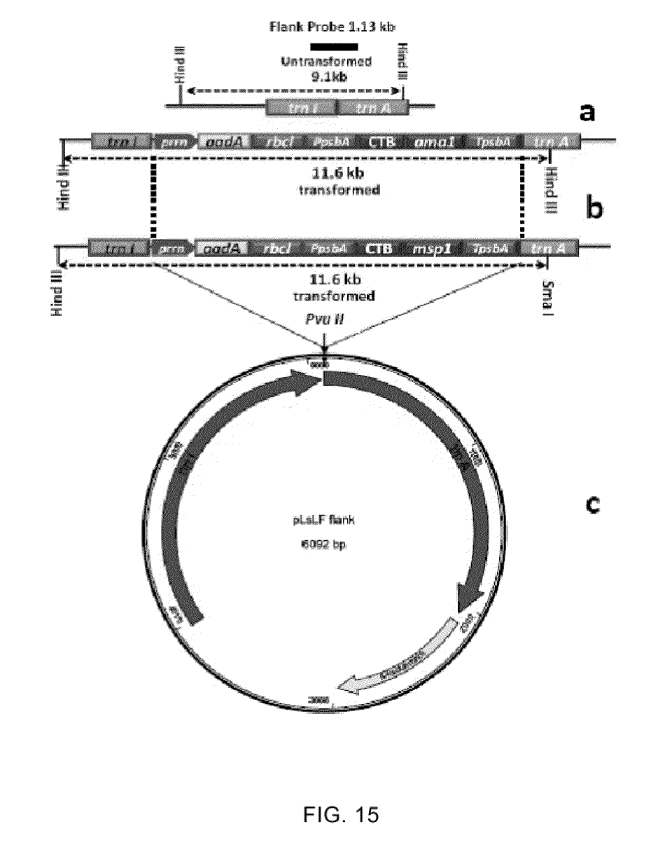

Lettuce chloroplast transformation vector pLsDV CTB-AMA1 and pLsDV CTB-MSP1 was constructed using endogenous regulatory elements from lettuce. The Prm:aadA:rbcL selectable marker gene cassette contained rrn promoter and rbcL 3′ untranslated region (UTR) amplified from lettuce chloroplast genome and cloned into pLsDV. The psbA: CTB-AMA1:psbA and psbA: CTB-AMA1: psbA expression cassette contained native psbA 5′ and 3′UTR's of lettuce. The selectable marker gene cassette and CTB-AMA1 and CTB-MSP1 fused genes expression cassette were cloned in tandem and inserted into the pUC-based lettuce targeting vector (pLsDV; FIG. 15, C) at a unique puv II restriction site (position 103 002) between trnI and trnA resulting the final lettuce chloroplast vector: pLsDV CTB-AMA-1 and pLsDV CTB-MSP-1 as described by Verma and Daniell (2007); Verma et al., (2008).

### Materials and Methods

Plant transformation and selection of transgenic plants Seeds of Lactuca sativa var. Simpson elite (New England Seed Co., Hartford, Conn., USA) were surface sterilized in a 30% Chlorox® commercial bleach and 0.01% Tween-20 for 5 min, rinsed five times in sterile water and plated on half strength Murashige and Skoog (MS) medium solidified with 6 g L-1 Phytablend® (Caisson, North Logan, Utah, USA). 21 d young, fully expanded leaves (˜4 cm²) were placed, adaxial side up, on antibiotic-free lettuce regeneration (LR) medium containing MS basal salts, thiamine-HCl 10 mg L-1, myo-inositol 100 mg L⁻¹, glycine 2 mg L⁻¹ naphthaleneacetic acid (NAA) 0.1 mg L⁻¹ and 6-benzylaminopurine (BAP) 0.2 mg L⁻¹. The leaves were bombarded with 0.6-μm gold particles coated with plasmids pLs DV-CTB-AMA1.DV; pLs DV CTB-MSP1 [FIGS. 15(a) and (b)], using particle delivery system PDS-1000/He (Bio-Rad, Hercules, Calif., USA) employing 900 psi rupture discs and a target distance of 6 cm as described by Verma et al. (2008). Bombarded leaves were incubated in the dark at 25° C. for 2 days prior to the explants of 0.5-cm² pieces. Explants were placed adaxial side down, on to LR medium containing 50 mgL⁻¹ spectinomycin dihydrochloride. Primary regenerants were screened by PCR for the transplastomic event, and positive shoots were subjected to an additional regeneration cycle on LR medium. Following the second regeneration, the shoots were rooted in lettuce development (LDS) containing half-strength MS basal salt, thiamine-HCl 10 mgL⁻¹, myo-inositol 100 mgL⁻¹ and spectinomycin 100 mgL⁻¹. The plantlets were rooted in Jiffy® peat pots and acclimatized in biodome before transfer to the glasshouse for seed production. TO seeds were harvested when the seed turned black and pappus become visible. Seeds were air dried at room temperature prior to germination. Sterile seeds (20-40) were plated on MS medium containing 100 mg L⁻¹ spectinomycin to confirm cytoplasmic maternal inheritance.

PCR analysis to confirm transgene integration into lettuce chloroplast genome DNA from transgenic and wild type plants was extracted using Qiagen DNeasy Plant Mini Kit (Qiagen, Valencia, Calif.). PCR was performed using the peltier thermal cycler PTC-100 (Bio-Rad, Hercules, Calif., USA). The integration of aadA gene into the chloroplast genome was assessed by using 16s forward primer (5′CAGCAGCCGCGGTAATACAGAGGA 3′; SEQ ID NO:9) that anneal with the native chloroplast genome and an aadA-gene specific primer (5′CCGCGTTGTTTCATCAAGCCTTACG 3′) that anneals with aadA gene. PCR reactions contained 1×Taq buffer, 0.5 mM dNTPs, 0.2 mM 16s forward primer, 0.2 mM 3M reverse primer, 0.05 units μl-1 Taq polymerase, 0.5 mM MgCl₂ and template DNA. Samples were run for 30 cycles as follows 94° C. for 1 min, 55° C. for 1 min and 72° C. for 3 min with a 5 min ramp up at 95° C. at the beginning of the PCR cycles and a 72° C. hold for 10 min at the end of 30 cycles. PCR products were separated on 0.7% agarose gels.

### Confirmation of Transgene Integration and Homoplasmy

Total plant DNA was digested with Hind III at 37° C. for 1 h and run on a 0.7% agarose gel at 65 V for 5-6 h. Prior to transfer the gel was depurinated (0.25 N HCl for 15 min) and denatured (0.4N NaOH for 20 min), transferred DNA to nitrocellulose membrane overnight. Membrane with transferred DNA was rinsed briefly in 2×SSC (0.3M NaCl, 0.03 M sodium acetate), dried on filter paper and crosslinked the DNA to membrane using GS GeneLinker™ (Bio-Rad, Hercules, Calif., USA). The flanking probe was prepared by digesting the pLsDV basic targeting vector containing only trnI and trnA flanking sequences with BamHI to yield a 1.1 kb fragment that was gel purified using a Qiaquick Gel Extraction Kit (Qiagen Inc. Valencia, Calif., USA). The probe was labeled by incubating with α³²P(-dCTP) and Ready-To-GoT DNA Labelling Beads (GE Healthcare, El Paso, Tex.) at 37° C. for 1 h. The excess ³²P was removed using the ProbeQuant G-50 Micro columns (Amersham, Arlington Heights, Ill.). Then the labeled probe was hybridized with nitrocellulose membrane using Stratagene QUICK-HYB hybridization solution and protocol (Stratagene, La Jolla, Calif., USA). Radiolabelled membranes were exposed to blue sensitive autoradiography flim BX (MIDSCI, St. Louis, Mo.) at −80° C. for 16 h.

### Protein Extraction

Fresh and healthy leaves from confirmed homoplasmic plants were used to extract protein. Leaf tissue was ground in liquid nitrogen with a mortar and pestle and approximately 100 mg of powdered leaf was taken into 300 μl of plant extraction buffer (PEB) [100 mM NaCl, 20 mM EDTA, 200 mM Tris-HCl, 0.1% SDS, 0.5% Tween-20, 14 mM 2-mercaptoethanol, 0.1 mM phenylmethanesulfonyl Fluoride, 1 tablet of Complete-protease inhibitor cocktail (Roche Diagnostics, UK) and 100 mM Dithiothreitol]. Prior to the centrifugation 100 μl of homogenate was taken in a pre-chilled microcentrifuge tube. The remaining plant extract was then centrifuged for 5 min at 10,000×g to pellet the insoluble plant material. The supernatant was divided into three portions and stored at −80° C. The pellet was resuspended in 300 μl PEB to check the presence of sediment protein.

### Estimation of Total Soluble Protein

The total soluble protein (TSP) from homogenate of the leaf samples was estimated using the Bio-Rad Bradford Protein Assay (BBPA). Bovine Serum Albumin (BSA) was used as a protein standard to construct the standard curve. BSA dilutions ranging from 0.05 to 1.0 μg μl⁻¹ with water and plant dilutions of 1:10 and 1:20 were made in water. 10 μl of the different concentrations of BSA standards and plant samples were loaded in replicate wells in a 96 well microtiter plate. One part of the concentrated BBPA dye was added to four parts of distilled water and filter sterilized. The wells with the standards, samples, and extraction buffer (for blanks) were loaded with 200 μl of the diluted BBPA dye. The plate was incubated at room temperature for 5 minutes and the absorbance was measured at 595 nm using a micro-plate reader (Model 680, Bio-Rad, Hercules, Calif., USA).

### Immunoblot Analysis

After estimation of TSP, 10 μg from sample was separated by 12% sodium dodecylsulfate-polyacrylamide gel electrophoresis (SDS-PAGE) and transferred to nitrocellulose membranes for immunoblotting, according to Kumar and Daniell (2004). The protein separated by SDS-PAGE gel was transferred to nitrocellulose membrane by electroblotting and the membrane was blocked overnight with 3% non-fat dry milk. To detect CTB-AMA1 and CTB-MSP1 fused proteins, blots were incubated with 1:3000 rabbit anti-CTB primary polyclonal antibody (Sigma, St. Louis, Mo., USA) followed by 1:5000 HRP-conjugated donkey anti-rabbit secondary antibody (Southernbiotech, Birmingham, Ala., USA). A SuperSignal®) West Pico chemiluminescence substrate Kit (Pierce, Rockford, Ill., USA) was used for autoradiographic detection.

Estimation of CTB-AMA1 and CTB-MSP1 Fusion Protein from Lettuce

ELISA was performed in duplicate in a 96-well, flat-bottomed, microtiter plates (Costar®) 96-Well EIA/RIA (Costar, Corning Inc. NY, USA) coated with purified CTB standard (Sigma, St. Louis, Mo., USA) and homogenate of leaf extract. The standard and homogenate was diluted in coating buffer (15 mM Na₂CO₃, 35 mM NaHCO₃, 3 mM NaN₃, pH 9.6). 100 μl of standard ranging from 6.25 to 200 ng ml⁻¹ and sample dilutions 1:10,000, 1:20,000 was used to coat 96-well microtiter plates, incubated at 4° C. overnight. The background was blocked with 3% fat-free milk in phosphate buffer saline and 0.05% Tween-20 (PBST), incubated at 37° C. for 1 h and washed three times with PBST and water alternatively. 100 μl of anti-CTB polyclonal antibody (Sigma, St. Louis, Mo., USA) diluted (1:3000) in PBST with 3% milk was loaded into wells, incubated at 37° C. for 1 h. The wells were then washed with PBST and water three times and loaded 100 μl of diluted (1:5000) HRP-conjugated mouse anti-rabbit (Southernbiotech, Birmingham, Ala., USA), incubated at 37° C. for 1 h, washed the wells with PBST and water. 100 μl of 3.3′, 5.5′-tetramethy benzidine (TMB) substrate was loaded to wells, incubated at room temperature for 10-15 min. The reaction was terminated by addition of 50 μl of 2N H2SO4. Microtiter plate was read at 45 nm using a plate reader (Model 680, Bio-Rad, Hercules, Calif., USA).

### Results

Lettuce vectors pLsDV CTB-AMA-1 and pLsDV CTB-MSP-1

The pLsDV CTB-AMA-1 (8.6 kb) and pLsDV CTB-MSP1 (8.6 kb) was cloned as described by Verma and Daniell (2007); Verma et al. (2008) to transform lettuce chloroplasts. The expression cassette includes a chloroplast operon promoter, ribosome-binding sequence (GGAGG; SEQ ID NO:10), selectable marker gene (aadA), 3′ UTR, 5′ UTR (PpsbA), CTB-ama1 or CTB-msp1 and its 3′ UTR (TpsbA). The constitutive 16s ribosomal operon promoter, that can be recognized by both plastid and nuclear encoded RNA polymerases, was used to drive the transcription of the aadA and CTB fused AMA1 and MSP1 genes. The aadA gene conferring spectinomycin resistance was used to select transgenic shoots. The CTB-AMA-1 and CTB-MSP-1 genes coding for apical membrane antigen-1 and merozoite surface protein-1 was regulated by the psbA 5′ and 3′ elements. The 5′-UTR from psbA, including its promoter, was used for the transcription enhancement of the CTB-AMA-1 and CTB-MSP-1 and it also serve for translation enhancement as it has several sequences of ribosomal binding sites. It acts as a scaffold for the light regulated enhanced translation for protein synthesis. The 3′psbA-UTR conferred transcript stability. The vector was delivered into chloroplast using biolistic method targeting trnI and tmA intergenic spacer region of the chloroplast genome. The selectable marker gene aadA and the fusion gene CTB-ama1 and CTB-msp1 were integrated into the chloroplast genome by homologous recombination.

Transgene Integration into the Plastome

### PCR Analysis

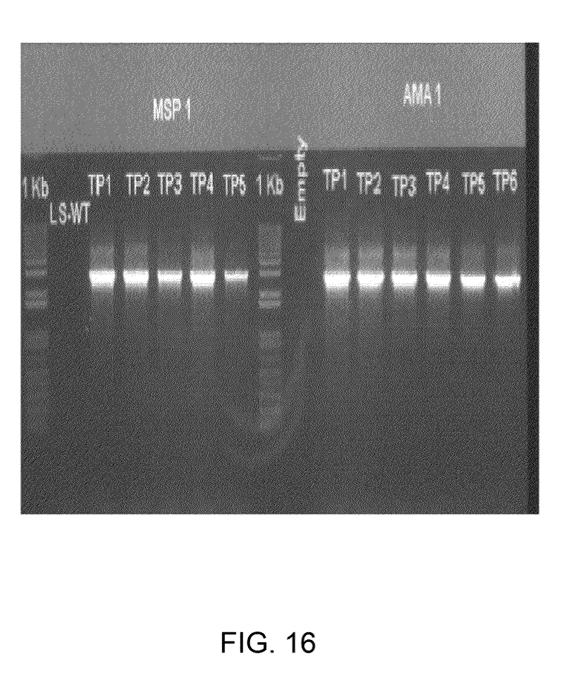

After bombardment, excised leaf disc were grown on LR medium supplemented with 50 μg ml⁻¹ of spectinomycin for selection. Five to six spectinomycin resistant shoots were observed when ten lettuce leaves were bombarded after three weeks of selection. These spectinomycin resistant shoots were screened for chloroplast transformation by PCR with two specific primers, 16s forward and 3M reverse primers. All six resistant shoots from pLsDV CTB-AMA-1 and five from pLsDV CTB-MSP-1 showed PCR positive for chloroplast transgenic lines (FIG. 16).

### Southern Analysis to Confirm Chloroplast Integration and Homoplasmy

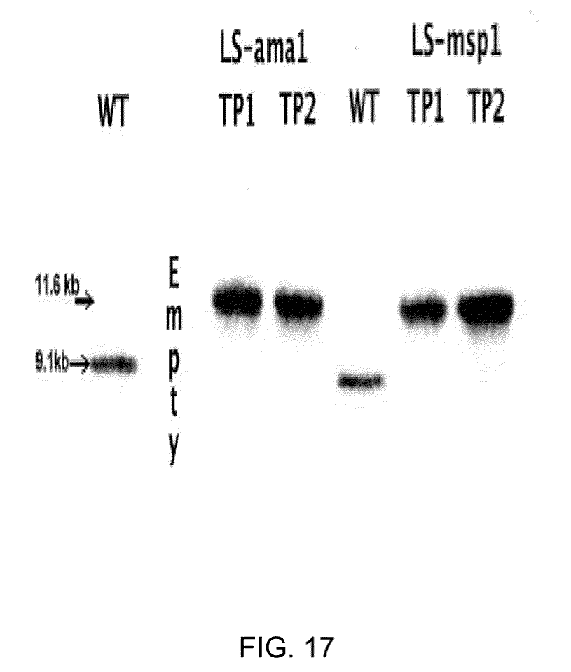

PCR positive plants were further examined by Southern analysis in order to confirm site-specific integration and to determine whether they were homoplasmic or heteroplasmic. Homoplasmy is achieved when all the copies of the genome within the chloroplast have stably integrated the transgenes. Total plant DNA from transformed and wild type plants were extracted and 0.5 μg of DNA from each sample was digested with Hind III which generated 9.1 kb in wild type, 11.6 kb in pLsDV CTB-AMA-1 and 11.5 kb in pLsDV CTB-MSP-1 transgenic lines when hybridized with a 1.13 kb trnI-trnA flank probe prepared from pLsDV-flank basic vector (FIG. 15 C). All pLsDV CTB-AMA-1 and pLsDV CTB-MSP-1 transgenic plants from the third round of selection showed homoplasmy (FIG. 17).

### CTB-AMA1 and CTB-MSP1 Expression in Lettuce Chloroplasts

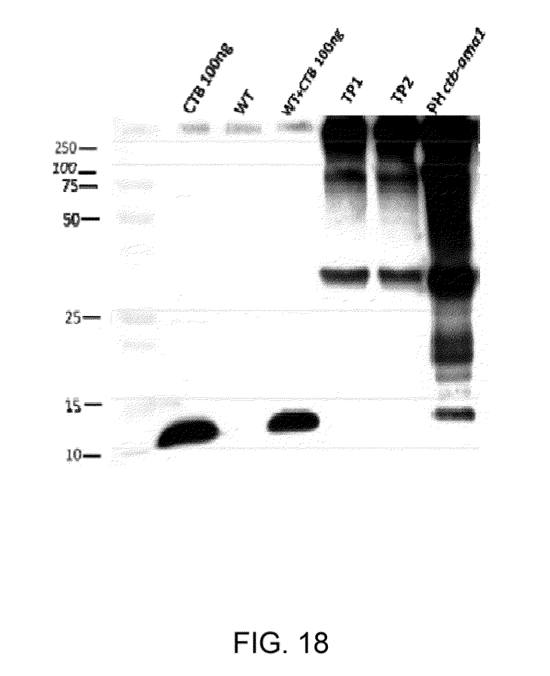

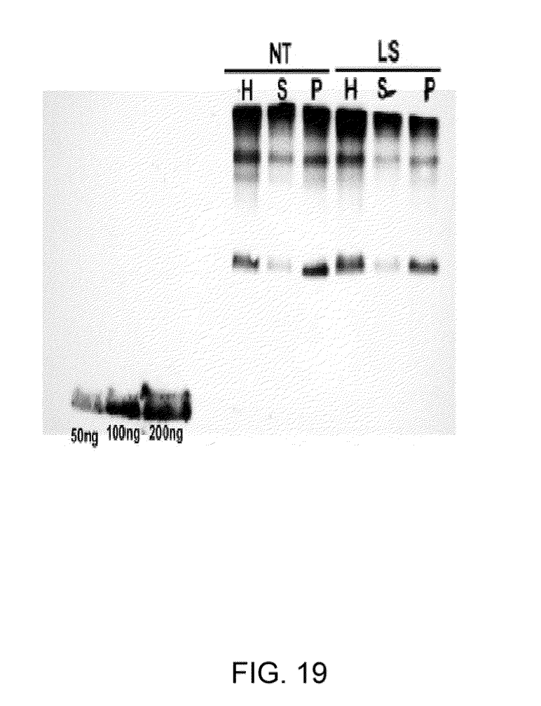

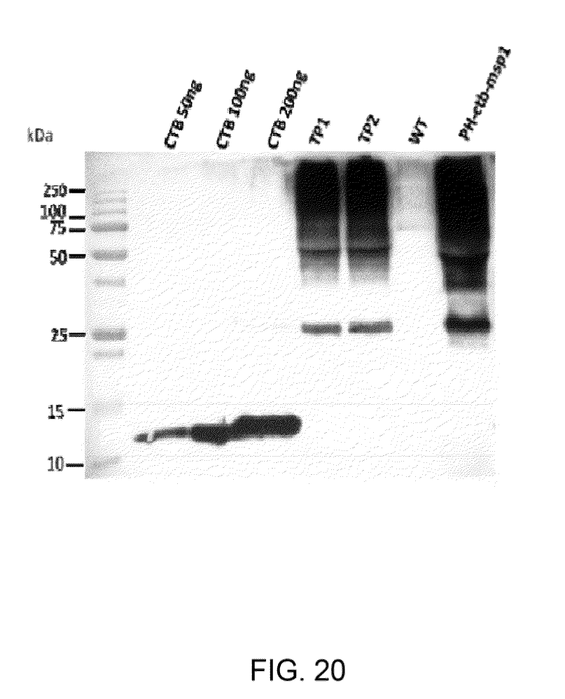

Total protein was extracted in the plant extraction buffer from 100 mg of leaf tissue. Immunoblots were performed on transgenic lines containing CTB-AMA-1 and CTB-MSP-1 transgene. Immunodetection with CTB polyclonal antibody showed 27.5 kDa of CTB fused polypeptide on CTB AMA-1 blots (FIGS. 18 and 19)) and a 23 kDa of CTB fused polypeptide on CTB MSP-1 blots (FIG. 20). The formation of dimers, trimers, tetramers and pentamers of the CTB-AMA1 and CTB-MSP1 fusion protein was observed. A large amount of protein could be detected in pellet (FIG. 19). Therefore, quantification of CTB-AMA-1 and CTB-MSP-1 was performed using homogenate.

### Quantification of Chloroplast Expressed CTB-AMA1 and CTB-MSP1

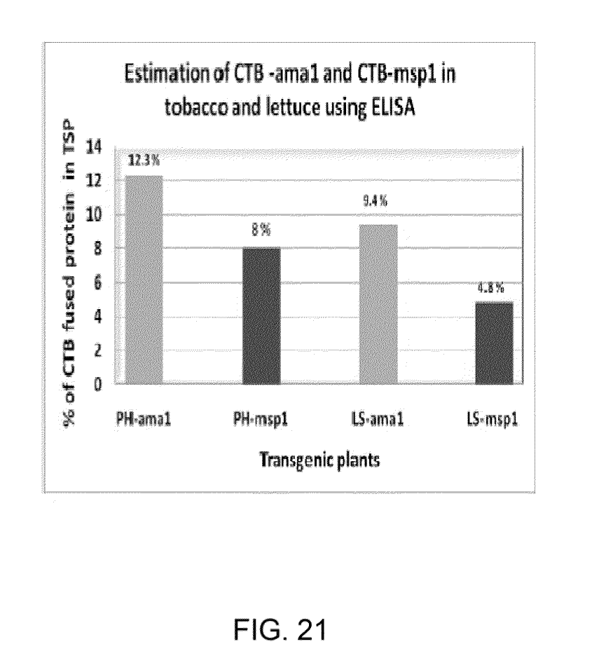

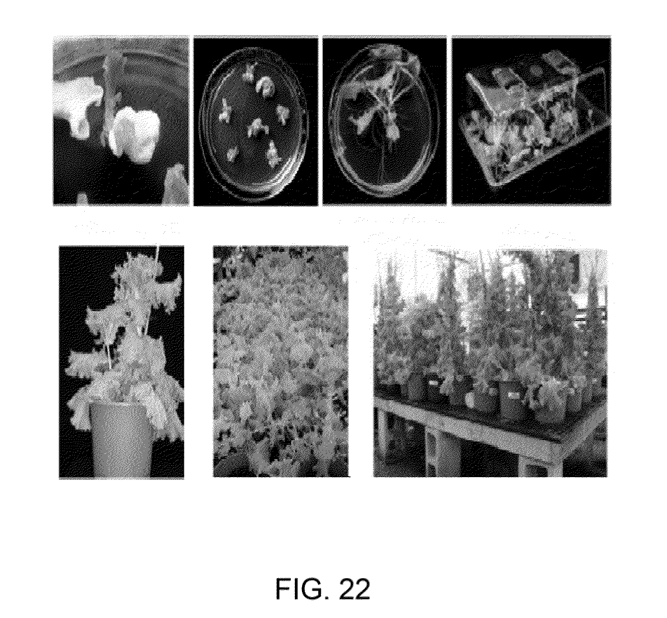

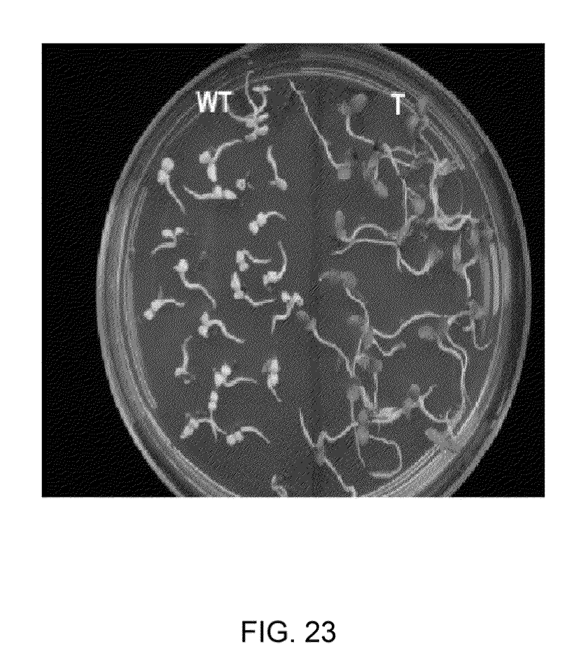

ELISA was performed to quantify the chloroplast derived CTB-AMA-1 and CTB-MSP-1 antigen in the homogenate of lettuce and tobacco. A standard curve was obtained with the purified bacterial CTB. Lettuce plants transformed with pLsDV CTB-AMA-1 and pLsDV CTB-MSP-1 constructs expressed CTB-AMA-1 and CTB-MSP-1. The CTB-AMA-1 and CTB-MSP-1 protein expression level of tobacco matured leaves reached 12.3% and 8% of the TSP respectively. Whereas, in lettuce the CTB-AMA-1 and CTB-MSP-1 protein expression level reached 9.4% and 4.8% of the TSP respectively under the green-house growth conditions (FIG. 21); (Table 4). A 100 mg of matured leaves yielded 3.33 mg and 1.56 mg of CTB-AMA-1 fused protein in tobacco and lettuce respectively. 100 mg of matured leaves yielded 2.16 mg and 0.66 mg of CTB-MSP-1 antigen was produced in transformed tobacco and lettuce respectively (see Table 4).

### Discussion

Currently, there is no licensed vaccine for the prevention of malarial disease despite the vast knowledge of genomics and proteomics of the malaria parasite (Sharma and Pathak 2008). The need for a malarial vaccine is imperative because the global burden of the disease is increasing due to drug resistance, resistance of mosquitoes to insecticides, ineffective control measures, re-emergence of the disease, and increased tourism. Malaria vaccine research has investigated several vaccine candidates in clinical trials but with disappointing results in low titers and poor efficacy. Research has lead to the discovery of AMA-1 and MSP-1, which are two of the leading asexual blood-stage malarial vaccine candidate antigens.

In the present work, the malarial genes, for AMA-1 and MSP-1, were successfully amplified via PCR along with the transmucosal carrier, CTB. CTB was included in the present study because of it's potential adjuvant activity on the immune system and possibility of performing as an oral immunogen (Li and Fox 1996). The fusion CTB-malarial gene cassettes were constructed in the pBSK+ vector.

Malaria antigens have been expressed in several systems, for example, in E. coli (Sachdeva, Mohmmed et al. 2006), yeast (Gozalo, Lucas et al. 1998), and mammalian cells (Burghaus, Gerold et al. 1999); however, disadvantages such as incorrect folding, low yields, and expensive production and purification procedures have persisted. Plants could be considered as an alternative in vaccine development because they can reduce cost due to the expense of purification, processing, cold storage, and delivery. In the present work, a major purpose of expressing the CTB-malarial antigens in the chloroplast system was to develop a safer and more effective malarial vaccine. By using the flanking sequences trnI and trnA, the chloroplast pLD-UTR vector with the CTB-malarial cassette was integrated into the chloroplast genome via homologous recombination. The pLD-UTR vector contains the aadA, which encodes for the enzyme aminoglycoside 3′ adenyltransferase and transgenic shoots were selected due to their resistance to spectinomycin (Svab and Maliga 1993) (Verma and Daniell 2007). 3P/3M and 5P/2M primers and PCR confirmed the integration of the gene cassette into the chloroplast genome. The positive transformants were subjected to second and third round of selection to remove any remaining wild type cells. The transgenic plants were confirmed to be homoplasmic (presence of only transformed genomes) by southern blot analysis with a flanking sequence probe.

The expression of CTB FC AMA-1 and CTB MSP-1 was driven by the psbA 5′UTR in transgenic lines and confirmed by immunoblot. The proteins were observed at 27.5 kDa in CTB FC AMA-1 and 23 kDa in CTB MSP-1 in both the insoluble (pellet) and soluble (supernatant) fractions as seen in FIG. 7. Along with the expression of the CTB-malarial monomer, the immunoblot displayed other forms, such as dimers, trimers, tetramers, and pentamers. The expression levels in mature leaves of CTB FC AMA-1 and CTB MSP-1 protein reached 6.3-9.5% and 1.4-2%, respectively, as shown in FIG. 8. The expression level of CTB FC AMA-1 and CTB MSP-1 is comparable to CTB-Pins in lettuce of 1.8% TSP (Ruhlman, Ahangari et al. 2007) but higher levels were seen with 14.8% accumulation of F1-V (Arlen, Singleton et al. 2008), 14% of anthrax protective antigen (Koya, Moayeri et al. 2005), and 16% of CTB-Pins in tobacco (Ruhiman, Ahangari et al. 2007). Although lower levels of expression were observed with the chloroplast-derived CTB-malarial proteins in tobacco it still provides a sufficient level of expression to proceed with animal or preclinical studies (Ruhlman, Ahangari et al. 2007).

The antigen administered to the mice via subcutaneous injection was enriched rather than purified because the CTB-malarial protein did not contain an epitope tag that would facilitate purification. CTB is known to bind to immobilized nickel ions (Dertzbaugh and Cox 1998) and this lead to enriching the CTBmalarial antigen in plant extracts using nickel beads. The CTB-malarial antigens were successfully enriched with nickel beads and to ensure adequate amounts for immunization studies talon enrichment was used. The oral-deliverable plant material was administered to mice by oral gavage.

The immunization schedule to compare subcutaneous injection versus oral delivery of chloroplast-derived was followed as indicated in Table 1. Following post-immunization, serum was collected from the five different bleeds and tested for anti-PfMSP1₁₉ antibodies by a capture ELISA with MRA-56 PfMSP1₁₉ protein. Immunization studies confirmed the chloroplast-derived malarial antigens were immunogenic in groups 5 and 6. The titers of groups 3 and 4 were not completed because of the lack of AMA-1 protein. Also, the titers of groups 7 and 8 were not completed because of the lack of adequate amounts of MSP-1 protein. Mouse titers ranged from 0-1:50,000 with higher titers in bleeds #3, #4, and #5 and higher titers in group 5 CTB MSP-1 (subcutaneous injection) versus group 6 (oral delivery). Four mice showed undetectable titers (one mouse in group 5 and three mice in group 6) with the ELISA and MRA-56 PfMSP1₁₉ protein (Table 2). Higher titers may have been observed in group 5 versus group 6 because of the difficulty in delivering an exact amount of antigen in oral gavage for every mouse. IFA and immunoblots confirmed that sera from immunized mice recognized native parasite and native parasite protein, respectively.

To investigate if mice elicited AMA-1 and MSP-1 antibodies after immunization and if they prevent parasite invasion into RBCS, an in vitro parasite inhibition assay was performed. If anti-malarial antibodies are present in sera collected from immunized mice there should be a decrease in parasitemia after the invasion assay. In the control groups, blank wells (no sera added); sera from mice immunized with non-transgenic plant material or alhydrogel alone, and mice receiving no immunization, similar levels of parasitemia were observed. Inhibition was observed with sera collected from mice immunized with injectable CTB-malaria antigen and oral delivery with the highest percent of inhibition was found in mice receiving subcutaneous injection of CTB MSP-1. Unpurified antibody (MRA-35 PfMSP1₁₉) collected from mice immunized with yeast secreted PsMSP1₁₉ resulted in 53% of inhibition that was comparable with mice immunized with chloroplast-derived CTB-malaria antigens.

### CONCLUSIONS

Malaria is a prominent, vector-borne parasitic disease and severe public health problem globally, and is especially prevalent in poor, developing countries. There is a great need to create a low cost human malarial vaccine with the elimination of laborious purification techniques and technical skills.

The present work discloses that two leading blood stage malarial vaccine candidates AMA-1 and MSP-1 were constructed in a fusion cassette with CTB. The CTB-malarial antigens were expressed in tobacco plants via plastid transformation and accumulated from moderate to high levels in CTB FC AMA-1 and CTB-MSP-1, about 9.5% and 2% of the total soluble protein, respectively. The chloroplast-derived CTB-malarial proteins were administered to mice by one of two routes, either by subcutaneous injection or by oral gavage. The immunogenicities of the antigens were determined to be in the range of 1:100-1:50,000 and higher titers found in mice from group 5 versus group 6. To maximize the titers in group 6 mice, an improved approach needs to be developed for delivering an equal amount of antigen to every mouse. Sera collected from mice immunized with both CTB FC AMA-1 and CTB MSP-1 were found to recognize native parasite and native parasite protein in both IFA and immunoblot analyses, indicating that anti-AMA-1 and anti-MSP-1 were elicited in the immunized mice. Anti-malarial antibodies were found to inhibit parasite invasion of erythrocytes with highest percent of inhibition occurring in mice immunized with CTB MSP-1 by subcutaneous injection. An appropriate animal model needs to be established before in vivo challenge and protection can be investigated. Results of these investigations may lead further experimentation in malarial vaccine development with other malarial antigens and, particularly, with transformed edible crops.

In view of the detailed description set forth above, the summary of the invention and the description of the drawings, the present invention discloses the following useful, novel and nonobvious features.

The invention provides a method of producing malaria antigens in a plant, the method comprising stably transforming the plant by inserting into its plastid genome a nucleic acid sequence encoding and operable to express a malaria antigenic polypeptide selected from AMA-1, MSP-1 or both.

The method of the invention includes an embodiment wherein the nucleic acid sequence further comprises encoding a fusion protein consisting essentially of cholera toxin B subunit and the malaria antigenic polypeptide.

In the method of the invention, the plant may be an edible plant such as lettuce. However, the plant may also be a species of the genus Nicotiana and, particularly, a variety of Nicotiana tabacum.

The invention includes the plant stably transformed according to the method of the invention and its cuttings, seeds and progeny. A preferable plant and method include wherein the operable expression is constitutive.

The invention further includes a method of treating a host susceptible to malaria, the method comprising administering to the host the malaria antigenic polypeptides produced according to claim 1 by a route effective for eliciting an antibody response. In this method, the stably transformed plant is preferably edible and the route of administration is by ingestion of the plant or part thereof.

Also included in the disclosed invention is an expression cassette effective for stably transforming a plant plastid genome to express one or more malaria antigenic polypeptides, the cassette comprising a nucleic acid sequence including two untranslated flanking regions homologous to parts of and effective for integrating into the plastid genome, and between the flanking regions a region encoding a malaria antigenic polypeptide selected from AMA-1, MSP-1 and combinations thereof, a region encoding a marker conferring resistance to a selective agent and a promoter region effective for constitutive expression of at least the malaria antigenic polypeptide and the resistance marker.

The expression cassette preferably comprises between the flanking regions a region encoding cholera toxin B subunit, such that an expressed malaria antigenic polypeptide is a fusion polypeptide therewith. Also intended within the scope of the invention is a fusion polypeptide expressed by the cassette, in a form purified from the transformed plant, as well as the plant containing the plastid genome stably transformed with the cassette, and its cuttings, seeds and progeny.

Part of the invention is an oral vaccine effective in raising malaria antibodies in a susceptible host, the vaccine comprising leaf material from an edible plant containing plastids stably transformed to constitutively express a fusion polypeptide consisting essentially of cholera toxin B subunit and a malaria antigenic polypeptide selected from AMA-1, MSP-1 or both. A method of treating a host susceptible to malaria comprises orally administering the described vaccine.

A method of making a malaria vaccine is additionally disclosed and claimed, the method comprising stably transforming a plant by inserting into its plastid genome a nucleic acid sequence encoding and operable to constitutively express a malaria antigenic polypeptide selected from AMA-1, MSP-1 or both; harvesting the stably transformed plant in whole or in part; purifying the expressed malaria antigenic polypeptide from the harvested plant; and packaging the purified antigenic polypeptide under sterile conditions in an amount for a predetermined dosage. In this method as well, the plant is preferably a species of the genus Nicotiana and most preferably a variety of the species Nicotiana tabacum.

Yet another aspect of the invention includes a method of making an oral malaria vaccine, the method comprising stably transforming an edible plant by inserting into its plastid genome a nucleic acid sequence encoding and operable to constitutively express a malaria antigenic polypeptide selected from AMA-1, MSP-1 or both; harvesting the stably transformed edible plant or parts thereof; and packaging the harvest for oral consumption. In this method, the harvest may be packaged in dried form.

Accordingly, in the drawings and specification there have been disclosed typical preferred embodiments of the invention and although specific terms may have been employed, the terms are used in a descriptive sense and not for purposes of limitation. The invention has been described in considerable detail with specific reference to the illustrated embodiments. It should be apparent to the skilled, however, that various modifications and changes can be made within the spirit and scope of the invention as described in the foregoing specification and as defined in the appended claims.

TABLE 1
Schedule of immunization of Mice

| | | | | | Amount of | |
|---|---|---|---|---|---|---|
| | Immunization | Immunization | Route of | | Material or Antigen | |
| Group | Type | Sample | Administration | Schedule | Delivered Per Dose | |
| 1 | Boost | WT PH | Oral | Days 10, 17, 24, | 0 | ug |
| | | | | 31, 37, 45, 52, | | |
| | | | | 59, 150, 157, 189 | | |
| 2 | Boost | AlH | s.c. | Days 13, 27, 43, | 0 | ug |
| | | | | 55, 150, 189 | | |
| 3 | Prime | enCtb FC AMA-1 | s.c. | Day 0 | 25 | ug |
| | Boost | enCtb FC AMA-1 | s.c. | Days 13, 27, 43, | 25 | ug |
| | | | | 55, 150, 189 | | |
| 4 | Prime | enCtb FC AMA-1 | s.c. | Day 0 | 25 | ug |
| | Boost | Ctb FC AMA-1 | Oral | Days 10, 17, 24, | 500 | mg*** |
| | | | | 31, 37, 45, 52, | | |
| | | | | 59, 150, 157, | | |
| | | | | 189, 220 | | |
| 5 | Prime | enCtb MSP-1 | s.c. | Day 0 | 25 | ug |
| | Boost | enCtb MSP-1 | s.c. | Days 13, 27, 43, | 25 | ug |
| | | | | 55, 150, 189 | | |
| 6 | Prime | enCtb MSP-1 | s.c. | Day 0 | 25 | ug |
| | Boost | Ctb MSP-1 | Oral | Days 10, 17, 24, | 500 | mg*** |
| | | | | 31, 37, 45, 52, 59, | | |
| | | | | 150, 157, 189 | | |
| 7 | Prime | enCtb FC AMA-1 | s.c. | Day 0 | 25 | ug |
| | | enCtb MSP-1 | | | | |
| | Boost | enCtb FC AMA-1 | s.c. | Days 13, 27, 43, | 25 | ug |
| | | enCtb MSP-1 | | 55, 150, 189 | | |
| 8 | Prime | enCtb FC AMA-1 | s.c. | Day 0 | 25 | ug |
| | | enCtb MSP-1 | | | | |
| | Boost | Ctb FC AMA-1 Ctb | Oral | Days 10, 17, 24, | 500 | mg*** |
| | | MSP-1 | | 31, 37, 45, 52, | | |
| | | | | 59, 150, 157, 189 | | |
| 9 | Nothing | Nothing | Nothing | Nothing | 0 | ug |

10 Mice Per Group: 90 mice Total
WT PH: non-transgenic tobacco plants
AlH: Alhydrogel (Adujvant)
Oral: oral gavage
s.c: subcutaneous injection
enCtb FC AMA-1: Enriched Ctb FC AMA-1
Ctb FC AMA-1: Transgenic tobacco plants expressing Ctb FC AMA-1
enCtb MSP-1: Enriched Ctb MSP-1
Ctb MSP-1: Transgenic tobacco plants expressing Ctb MSP-1
***ORAL DELIVERY: Leaf material not antigen

TABLE 2
Immunogenicity of a Malarial Antigen Using MSP-1 Protein.

| | MSP.1 IgG1 | MSP.1 IgG1 | MSP-1 IgG1 | MSP.1 IgG1 | MSP.1 IgG1 |
|---|---|---|---|---|---|
| Mouse # | Titers Bleed #1 | Titers Bleed #2 | Titers Bleed #3 | Titers Bleed #4 | Titers Bleed #5 |
| 5A1 | 0 | 0 | 0 | 500 | 1000 |
| 5A2 | 0 | 1000 | 10000 | 25000 | 50000 |
| 5A3 | 1000 | 1000 | 10000 | 12500 | 25000 |
| 5A4 | 0 | 1000 | 1000 | 500 | 1000 |
| 5A5 | 0 | 500 | 1000 | 500 | 1000 |
| 5B1 | 0 | 0 | 0 | 0 | 1000 |
| 5B2 | 0 | 100 | 1000 | 500 | 1000 |
| 5B3 | 0 | 0 | 0 | 0 | 0 |
| 5B4 | 0 | 250 | 10000 | 12500 | 25000 |
| 5B5 | 0 | 0 | 100 | 500 | 25000 |
| 6A1 | 0 | 0 | 0 | 0 | 0 |
| 6A2 | 0 | 0 | 100 | 500 | 1000 |
| 6A3 | 0 | 0 | 0 | 0 | 0 |
| 6A4 | 0 | 0 | 100 | 0 | 0 |
| 6A5 | 0 | 0 | 100 | 500 | 1000 |
| 6B1 | 0 | 0 | 500 | 0 | 0 |
| 6B2 | 0 | 100 | 1000 | 500 | 1000 |
| 6B3 | 100 | 100 | 500 | 500 | 1000 |
| 6B4 | 0 | 0 | 0 | 0 | 0 |
| 6B5 | 100 | 500 | 1000 | 12500 | 25000 |

ELISA detecton of anti-MSP-1 antibody titers from groups 5 and 6 serum samples collected from five bleeds.

TABLE 3
Parasitemia and Inhibition of Parasite Invasion

| Group | Parasitemia | Average | Inhibition |
|---|---|---|---|
| No Ab | 6.6-6.7% | 6.6% | — |
| MRA-35 PfMSP1-19 | 2.5-3.5% | 3.1% | 53.0% |
| Group 1 | 5.9-6.6% | 6.1% | 7.6% |
| Group 2 | 5.5-6.2% | 5.8% | 12.1% |
| Group 3 | 2.8-3% | 2.9% | 56.1% |
| Group 4 | 2.4-3.3% | 3.0% | 54.5% |
| Group 5 | 2.4-2.6% | 2.5% | 62.1% |
| Group 6 | 2.6-3.6% | 3.2% | 51.5% |
| Group 7 | 2.7-3.9% | 3.3% | 50.0% |
| Group 8 | 3.3-3.8% | 3.6% | 45.5% |
| Group 9 | 5.6-5.8% | 5.7% | 13.6% |

Calculation of Average Parasitemia in RBCs and Percent Inhibition after in Vitro Parasite Inhibition Assay. Synchronized 3D7 P. falciparum trophozoite-schizont stage culture (2% parasitemia and hematocrit) and no sera, MRA-35 PfMSP1-19 sera, and immunized mouse sera was incubated for 48 hours; the parasitemia was estimated and percent of inhibition was determined.

TABLE 4
quantification of CTB-AMA1 and CTB-MSP1 antigens in chloroplast transformed tobacco and lettuce

| | | | Amount of | Percentage of CTB | Amount of CTB AMA-1 |
|---|---|---|---|---|---|
| | | | transgene | fused protein (CTB | and CTB MSP-1 in |
| Transgenic tobacco | | TSP | protein | AMA-1 and CTB | per gram of |
| and lettuce | gene | μg μl⁻¹ | μg μl⁻¹ | MSP-1) in TSP | leaf tissue |
| Nicotiana tabaccum | CTB-ama1 | 9.0 | 1.11 | 12.3 | 3.33 mg |
| Nicotiana tabaccum | CTB-msp1 | 9.0 | 0.72 | 8.0 | 2.16 mg |
| Lactuca sativa | CTB-ama1 | 5.5 | 0.52 | 9.4 | 1.56 mg |
| Lactuca sativa | CTB-msp1 | 4.5 | 0.22 | 4.8 | 0.66 mg |

1. A method of producing malaria antigens in a plant, the method comprising stably transforming the plant by inserting into its plastid genome a plastid vector comprising an expression cassette containing at least one heterologous nucleic acid sequence coding for and operable to express a malaria antigenic polypeptide selected from AMA-1, MSP-1 or both and operably linked with control sequences positioned upstream from the 5′ end and downstream of the 3′ end of the heterologous nucleic acid sequence to provide expression of the heterologous nucleic acid sequence in the chloroplast genome of the plant, and flanking each side of the expression cassette plastid nucleic acid flanking sequences which are highly conserved in substantially all higher plants, containing a plastid origin of replication and derived from a transcriptionally active spacer region, whereby stable integration of the heterologous nucleic acid sequence into a target plant's plastid genome is effected by homologous recombination of the flanking sequences with complementary sequences in the plant's plastid genome and wherein said stable integration is directed into a transcriptionally active intergenic spacer region of said plastid genome.

2. The method of claim 1, wherein the nucleic acid sequence further comprises encoding a fusion protein consisting essentially of cholera toxin B subunit and the malaria antigenic polypeptide.

3. The method of claim 1, wherein the plant is an edible plant.

4. The method of claim 1, wherein the plant is a species of the genus Nicotiana.

5. The method of claim 1, wherein the plant is a variety of Nicotiana tabacum.

6. The plant stably transformed according to the method of claim 1 and its cuttings, seeds and progeny.

7. The method of claim 1, wherein the operable expression is constitutive.

8. A method of treating a host susceptible to malaria, the method comprising administering to the host the malaria antigenic polypeptides produced according to claim 1 by a route effective for eliciting an antibody response.

9. The method of claim 8 wherein the stably transformed plant is edible and the route of administration is ingestion of the plant or part thereof.

10. An expression cassette effective for stably transforming a plant plastid genome to express one or more malaria antigenic polypeptides, the cassette comprising a nucleic acid sequence including two untranslated flanking regions homologous to parts of and effective for integrating into the plastid genome, and between the flanking regions a region encoding a malaria antigenic polypeptide selected from AMA-1, MSP-1 and combinations thereof, a region encoding a marker conferring resistance to a selective agent and a promoter region effective for constitutive expression of at least the malaria antigenic polypeptide and the resistance marker.

11. The expression cassette of claim 10, further comprising between the flanking regions a region encoding cholera toxin B subunit, such that an expressed malaria antigenic polypeptide is a fusion polypeptide therewith.

12. The fusion polypeptide expressed by the cassette of claim 11, in a form purified from the transformed plant.

13. The plant containing the plastid genome stably transformed with the cassette of claim 10, and its cuttings, seeds and progeny.

14. An oral vaccine effective in raising malaria antibodies in a susceptible host, the vaccine comprising leaf material from an edible plant containing plastids stably transformed according to the method of claim 1 to constitutively express a fusion polypeptide consisting essentially of cholera toxin B subunit and a malaria antigenic polypeptide selected from AMA-1, MSP-1 or both.

15. A method of treating a host susceptible to malaria, the method comprising orally administering the vaccine of claim 14.

16. A method of making a malaria vaccine, the method comprising:
stably transforming a plant by inserting into its plastid genome a plastid vector comprising an expression cassette containing a heterologous nucleic acid sequence encoding and operable to constitutively express a malaria antigenic polypeptide selected from AMA-1, MSP-1 or both and operably linked with control sequences positioned upstream from the 5′ end and downstream of the 3′ end of the heterologous nucleic acid sequence to provide expression of the heterologous nucleic acid sequence in the chloroplast genome of a target higher plant, and flanking each side of the expression cassette plastid nucleic acid flanking sequences which are highly conserved in higher plants, containing a plastid origin of replication and derived from a transcriptionally active spacer region, whereby stable integration of the heterologous nucleic acid sequence into a target plant's plastid genome is facilitated by homologous recombination of the flanking sequences with complementary sequences in the target plastid genome and wherein said stable integration is directed into a transcriptionally active intergenic spacer region of said plastid genome:
harvesting the stably transformed plant in whole or in part;
purifying the expressed malaria antigenic polypeptide from the harvested plant; and
packaging the purified antigenic polypeptide under sterile conditions in an amount for a predetermined dosage.

17. The method of claim 16, wherein the plant is a species of the genus Nicotiana.

18. The method of claim 16, wherein the plant is a variety of the species Nicotiana tabacum.

19. A method of making an oral malaria vaccine, the method comprising:
stably transforming a higher plant by inserting into its plastid genome a plastid vector comprising an expression cassette containing a heterologous nucleic acid sequence encoding and operable to constitutively express a malaria antigenic polypeptide selected from AMA-1, MSP-1 or both and operably linked with control sequences positioned upstream from the 5′ end and downstream of the 3′ end of the heterologous nucleic acid sequence to provide expression of the heterologous nucleic acid sequence in the chloroplast genome of the target higher plant, and flanking each side of the expression cassette, plastid nucleic acid flanking sequences highly conserved in higher plants, containing a plastid origin of replication and derived from a transcriptionally active spacer region, whereby stable integration of the heterologous nucleic acid sequence into a target plant's plastid genome is facilitated by homologous recombination of the flanking sequences with complementary sequences in the target plastid genome and wherein said stable integration is directed into a transcriptionally active intergenic spacer region of said plastid genome;
harvesting the stably transformed edible plant or parts thereof; and
packaging the harvest for oral consumption.

20. The method of claim 19, wherein the harvest is packaged in dried form.


SEQUENCE LISTING (10 sequences)

SEQ ID NO: 1 - DNA, 32 bp, Artificial
  Primer
ccgctcgagcatatggctttgtcccatcccat

SEQ ID NO: 2 - DNA, 38 bp, Artificial
  Primer
ccgaattcggaccaggaccaatttcacaacaccaatga

SEQ ID NO: 3 - DNA, 28 bp, Artificial
  Primer
cggaattctttcatgttatcataagttg

SEQ ID NO: 4 - DNA, 42 bp, Artificial
  Primer
ataagaatgcggccgcttagttagaggaactgcagaaaatac

SEQ ID NO: 5 - DNA, 25 bp, Artificial
  Primer
aaaacccgtcctcagttcggattgc

SEQ ID NO: 6 - DNA, 25 bp, Artificial
  Primer
ccgcgttgtttcatcaagccttacg

SEQ ID NO: 7 - DNA, 24 bp, Artificial
  Primer
ctgtagaagtcaccattgttgtgc

SEQ ID NO: 8 - DNA, 24 bp, Artificial
  Primer
tgactgcccacctgagagcggaca

SEQ ID NO: 9 - DNA, 24 bp, Artificial
  Primer
cagcagccgcggtaatacagagga

SEQ ID NO: 10 - DNA, 5 bp, Artificial
  Ribosome binding sequence
ggagg